SEQUENCE LISTING (14 sequences)

SEQ ID NO: 1 - protein, 237 aa, synthetic construct
  REGION 1..237: note = hypothetical DegU protein screening sequence
MENKASRKIKVLLADDHQLFREGLKRILNMEDDLEVIGECGDGIQVLEFCNQNKPDVVLMDINMPIENGVEATERLREIFPDIKVIILSIHDDESYVFETLRKGASGYLLKDMEAESLINAIRSVVEGHAYIHPKVTGKLINQLRRMTYLDETGAASGAAKEAGVKFVAGDNNPLTRREAEVLRLMAEGKSNKMIGEFLFISEKTVKNHVSSILQKMEVDDRTQAVINSIKNGWVTL

SEQ ID NO: 2 - protein, 384 aa, synthetic construct
  REGION 1..384: note = hypothetical DegS protein screening sequence
MDFQADAIDRVIKNAIEVMEDSKYQIFEICESARQELESLNQELQQVLKETAETIDKVDQLELDYRRSRIRLTEVSRDFVRYKEEDIKQAYEKATQLQLDLMIYREKEMYLKARRDDLQKRVRNVEKSVERAETIGSQMNVVLEYLSGDLNQVTRILESAKNRQLIGLKIILAQEEERKRIAREIHDGPAQSLANLVLRTEIVERMLAKQEFEMVQDELVDLKGQVRSGLEEVRKIIFNLRPMALDDLGLVPTLRKYVQDFEEKTKIRTTFETRGKEHRLPSAMEAAIFRLVQEALSNAAKHAYATYVSVEITYQAQMVKIVVKDNGVGFDVELLEQKSKSHFGLIGMRERVELLEGRMEIESAEGQGTKIVIHIPINVEKRKE

SEQ ID NO: 3 - protein, 260 aa, synthetic construct
  REGION 1..260: note = hypothetical Spo0A protein screening sequence
MQKIKVCLADDNRELVNLLEEYISSQEDMEVIGVAYNGQECLELLEEKVPDVLILDIIMPHLDGLAVLERLREMNLSPQPKVIMLTAFGQEDVTKKAVELGASYYILKPFDMENLANRIRQVSGSSSVSSSSSSSNVVPEGKPKNLDASITSIIHEIGVPAHIKGYMYLREAITMVYNDIELLGSITKVLYPDIAKKFNTTASRVERAIRHAIEVAWSRGNIDSISSLFGYTVSMSKAKPTNSEFIAMVADKLRIEHKVS

SEQ ID NO: 4 - DNA, 20 bp, synthetic construct
  source 1..20: mol_type = other DNA
  misc_feature 1..20: note = artificial sequence for creating a degU Q218* mutation by changing nucleotide position 652 C to T
cagcagtattttgcaaaaaa

SEQ ID NO: 5 - DNA, 20 bp, synthetic construct
  source 1..20: mol_type = other DNA
  misc_feature 1..20: note = artificial sequence for creating a degU M220N (i.e. DegU D223*+M220N+E221G+V222G) mutation by inserting an A at nucleotide pos. 658
cagcagtattttgcaaaaaa

SEQ ID NO: 6 - DNA, 20 bp, synthetic construct
  source 1..20: mol_type = other DNA
  misc_feature 1..20: note = artificial sequence for creating a degS L99F mutation by changing nucleotide pos. 295 C to T
tgtgatgattttccgcgaga

SEQ ID NO: 7 - DNA, 20 bp, synthetic construct
  source 1..20: mol_type = other DNA
  misc_feature 1..20: note = artificial sequence for creating a spo0A A257V mutation by changing nucleotide pos. 770 C to T
taagctgagaattgagaaca

SEQ ID NO: 8 - DNA, 804 bp, Paenibacillus polymyxa
  source 1..804: mol_type = unassigned DNA
  misc_feature 1..804: note = DSM365 spo0A wild type
ttgcaaaaaattgaggtattgttggctgatgacaaccgggaatttacgaatttgcttgctgaatatatttccgatcaggaggatatggaagttacaggaatcgcctataacggcgaagaagtgctccaacgcatcgcagaatcccgcaacatacctgatgtacttattttagatattattatgcctcatctggatggtctcggtgtattggagcgtttaagagaaatgaacctgactccacagccgaaaatcattatgctgactgcattcggtcaagaaaatattacacagagggccgtacagctcggggcatcttattatattttgaagccgtttgacatggaagtgctggctaaccgtgttcgccaattagtggggccgcagttagttagcagcagtccggtgaccatttcatccatgcggtccaacgttgtacctatgggtaagacgaaaaacctggatgccagtatcacggccatcatacatgaaattggtgtaccagctcacatcaagggctatcaatatttacgcgaagccattacgatggtgtacaataatatcgaaattttgggagccatcaccaaaacattatatccggcaatcgccgaaaagttcaaaacgacggcatctcgcgtagaacgcgccattcgtcatgctatcgaggtagcatggacccgtggaaatatcgacagcatttcacacctgttcgggtacaccattaatatcagtaagtccaaaccaaccaattcagagttcatagcgatggtagcagataagctgagaattgagaacaaggtgtcctga

SEQ ID NO: 9 - DNA, 804 bp, synthetic construct
  source 1..804: mol_type = other DNA
  misc_feature 1..804: note = DSM365 spo0A A257V, pos. 770 C to T
ttgcaaaaaattgaggtattgttggctgatgacaaccgggaatttacgaatttgcttgctgaatatatttccgatcaggaggatatggaagttacaggaatcgcctataacggcgaagaagtgctccaacgcatcgcagaatcccgcaacatacctgatgtacttattttagatattattatgcctcatctggatggtctcggtgtattggagcgtttaagagaaatgaacctgactccacagccgaaaatcattatgctgactgcattcggtcaagaaaatattacacagagggccgtacagctcggggcatcttattatattttgaagccgtttgacatggaagtgctggctaaccgtgttcgccaattagtggggccgcagttagttagcagcagtccggtgaccatttcatccatgcggtccaacgttgtacctatgggtaagacgaaaaacctggatgccagtatcacggccatcatacatgaaattggtgtaccagctcacatcaagggctatcaatatttacgcgaagccattacgatggtgtacaataatatcgaaattttgggagccatcaccaaaacattatatccggcaatcgccgaaaagttcaaaacgacggcatctcgcgtagaacgcgccattcgtcatgctatcgaggtagcatggacccgtggaaatatcgacagcatttcacacctgttcgggtacaccattaatatcagtaagtccaaaccaaccaattcagagttcatagcgatggtagtagataagctgagaattgagaacaaggtgtcctga

SEQ ID NO: 10 - DNA, 723 bp, Paenibacillus polymyxa
  source 1..723: mol_type = unassigned DNA
  misc_feature 1..723: note = DSM365 degU wild type
atggaaaatcaagaaattagcaatgcacccattaaagttctcttagcggacgatcatcagttgttccgcgaaggacttaaacgtattttgaatatggaggacgacattgaggtcatcggagaatgtggcgatggtattcaagtgttggaattctgtaacgtagagaaaccggatattgttctgatggacattaatatgccaatcgaaaatggtgtagaggcaactgaaaaactgcgtgagatgttcccggatgtcaaagttatcattttatccattcatgatgatgaaagctatgtatttgagacgttgcgcaagggagccaatggttatctgttaaaggatatggaggccgagtccctcattaatgctattcgttcggttcatgaaggctatgcgtttatccatccgaaggtaacaggtaagctcattcagcaactccgccggatgacctacttgaatgaaacaggggctatggctgaaggtcatacgaaggaagccggcgtgaagtttgtcgcaggtgaaaataatccattgacccgtcgtgaagctgaagtattgcgtctgatggctgaaggtaagagcaacaagatgatcggtgaatatttattcattagtgagaaaacggtcaaaaaccatgtcagcagtattttgcaaaaaatggaggttgatgaccgtacacaagcagttattaactcaatcaaatatggatgggttacgctgtaa

SEQ ID NO: 11 - DNA, 724 bp, synthetic construct
  source 1..724: mol_type = other DNA
  misc_feature 1..724: note = DSM365 degU D223*+M220N+E221G+V222G, pos. 658 A to AA
atggaaaatcaagaaattagcaatgcacccattaaagttctcttagcggacgatcatcagttgttccgcgaaggacttaaacgtattttgaatatggaggacgacattgaggtcatcggagaatgtggcgatggtattcaagtgttggaattctgtaacgtagagaaaccggatattgttctgatggacattaatatgccaatcgaaaatggtgtagaggcaactgaaaaactgcgtgagatgttcccggatgtcaaagttatcattttatccattcatgatgatgaaagctatgtatttgagacgttgcgcaagggagccaatggttatctgttaaaggatatggaggccgagtccctcattaatgctattcgttcggttcatgaaggctatgcgtttatccatccgaaggtaacaggtaagctcattcagcaactccgccggatgacctacttgaatgaaacaggggctatggctgaaggtcatacgaaggaagccggcgtgaagtttgtcgcaggtgaaaataatccattgacccgtcgtgaagctgaagtattgcgtctgatggctgaaggtaagagcaacaagatgatcggtgaatatttattcattagtgagaaaacggtcaaaaaccatgtcagcagtattttgcaaaaaaatggaggttgatgaccgtacacaagcagttattaactcaatcaaatatggatgggttacgctgtaa

SEQ ID NO: 12 - DNA, 723 bp, synthetic construct
  source 1..723: mol_type = other DNA
  misc_feature 1..723: note = DSM365 degU Q218*, pos. 652 C to T
atggaaaatcaagaaattagcaatgcacccattaaagttctcttagcggacgatcatcagttgttccgcgaaggacttaaacgtattttgaatatggaggacgacattgaggtcatcggagaatgtggcgatggtattcaagtgttggaattctgtaacgtagagaaaccggatattgttctgatggacattaatatgccaatcgaaaatggtgtagaggcaactgaaaaactgcgtgagatgttcccggatgtcaaagttatcattttatccattcatgatgatgaaagctatgtatttgagacgttgcgcaagggagccaatggttatctgttaaaggatatggaggccgagtccctcattaatgctattcgttcggttcatgaaggctatgcgtttatccatccgaaggtaacaggtaagctcattcagcaactccgccggatgacctacttgaatgaaacaggggctatggctgaaggtcatacgaaggaagccggcgtgaagtttgtcgcaggtgaaaataatccattgacccgtcgtgaagctgaagtattgcgtctgatggctgaaggtaagagcaacaagatgatcggtgaatatttattcattagtgagaaaacggtcaaaaaccatgtcagcagtattttgtaaaaaatggaggttgatgaccgtacacaagcagttattaactcaatcaaatatggatgggttacgctgtaa

SEQ ID NO: 13 - DNA, 1161 bp, synthetic construct
  source 1..1161: mol_type = other DNA
  misc_feature 1..1161: note = DSM365 degS wild type
gtggactttcaagccgatatcatagaccgagtcattaaaaatgccatccaggtgatggagaacagcaaatatcagatgttcgaaatattggacacggcccggaccgagctggtcacgttaaatcaggagctccagagcgtcttgaaggaaacagcagaaacgatcgaaaaggtggaccagctggaaatgaactatcggcggtcccgcattcggttgactgaggtcagccgtgacttcgttcgctattcggaagaggatatcaagcaggcttatgagaaggcaacacagcttcaactcgatgtgatgattttccgcgagaaggaaatgtatctcaaagccagaagagatgatcttcaaaagcgggccaaaagtgttgaggcttctgtcgagcgcgccgaaaccatcggttcgcagatgggagtcgtgctggaatatttgtcgggcgaattgggacaagtgacgcggatcatcgaatcggctaaaaaccggcagtttattggtctgaaaattattttggctcaggaagaggagcgcaaacgcatatcccgtgaaattcacgatggacctgcgcagctccttgcgcatctagtgcttaggacggaaattgtggaaagaatgatcgccaagcaggaatttaagatggttcaggacgaaatagtagacttgaagaaacaggttcgctccagtcttgaggaaatgcgaaaggtcattttcaatttgcgtcctatggccctggatgacctgggacttattccgacgctccggaaatatgtgcaggattttgaagaaaaaacgaagattagatcgctttttgaaacaaggggcaaggaacaccgtctctcttccgcgatggaagcagccatttaccgtctgatccaggaagctttgaccaatgctgccaagcatgcctatcctacctatgtgcttgttgagattacctatcaggcgcagcttgtaaaaatcgtggtgcaggataatggtttgggctttaagccagagctttttcagcaaaaaagcaaagatcatgggcattttggtctgattggtatgcgggaaagggttgaactgctcgaggggagaatggagatcgaatcagctgagaatcaaggcaccaagatagtgattcatatcccaaccaacgtggaaaagggaaaggagtaa

SEQ ID NO: 14 - DNA, 1161 bp, synthetic construct
  source 1..1161: mol_type = other DNA
  misc_feature 1..1161: note = DSM365 degS L99F, pos. 295 C to T
gtggactttcaagccgatatcatagaccgagtcattaaaaatgccatccaggtgatggagaacagcaaatatcagatgttcgaaatattggacacggcccggaccgagctggtcacgttaaatcaggagctccagagcgtcttgaaggaaacagcagaaacgatcgaaaaggtggaccagctggaaatgaactatcggcggtcccgcattcggttgactgaggtcagccgtgacttcgttcgctattcggaagaggatatcaagcaggcttatgagaaggcaacacagcttcaattcgatgtgatgattttccgcgagaaggaaatgtatctcaaagccagaagagatgatcttcaaaagcgggccaaaagtgttgaggcttctgtcgagcgcgccgaaaccatcggttcgcagatgggagtcgtgctggaatatttgtcgggcgaattgggacaagtgacgcggatcatcgaatcggctaaaaaccggcagtttattggtctgaaaattattttggctcaggaagaggagcgcaaacgcatatcccgtgaaattcacgatggacctgcgcagctccttgcgcatctagtgcttaggacggaaattgtggaaagaatgatcgccaagcaggaatttaagatggttcaggacgaaatagtagacttgaagaaacaggttcgctccagtcttgaggaaatgcgaaaggtcattttcaatttgcgtcctatggccctggatgacctgggacttattccgacgctccggaaatatgtgcaggattttgaagaaaaaacgaagattagatcgctttttgaaacaaggggcaaggaacaccgtctctcttccgcgatggaagcagccatttaccgtctgatccaggaagctttgaccaatgctgccaagcatgcctatcctacctatgtgcttgttgagattacctatcaggcgcagcttgtaaaaatcgtggtgcaggataatggtttgggctttaagccagagctttttcagcaaaaaagcaaagatcatgggcattttggtctgattggtatgcgggaaagggttgaactgctcgaggggagaatggagatcgaatcagctgagaatcaaggcaccaagatagtgattcatatcccaaccaacgtggaaaagggaaaggagtaa

(19) United States
(12) Patent Application Publication
(10) Pub. No.: US 2024/0376424 A1
(43) Pub. Date: Nov. 14, 2024

(54) EXOPOLYSACCHARIDE PRODUCTION MICROORGANISMS AND USES THEREOF

(71) Applicant: BASF SE, Ludwigshafen am Rhein (DE)

(72) Inventors: Tobias MAY, Ludwigshafen (DE); Andrea HEROLD, Ludwigshafen (DE); Ingeborg HEUSCHKEL, Ludwigshafen (DE); Daniel Christoph HEINRICH, Ludwigshafen (DE); Heiko SENDROWSKI, Ludwigshafen (DE)

(21) Appl. No.: 18/682,052

(22) PCT Filed: Sep. 9, 2022
(86) PCT No.: PCT/EP2022/075122

§ 371 Date: Feb. 7, 2024

(30) Foreign Application Priority Data
Sep. 10, 2021 (EP) 21196088.5

(51) Int. Cl. C12N 1/20; A01N 63/22; C07K 14/195; C12N 15/74; C12P 19/04; C12R 1/12

(52) U.S. Cl. CPC C12N 1/205; A01N 63/22; C07K 14/195; C12N 15/74; C12P 19/04; C12R 2001/12

(57) ABSTRACT

Described herein are genes involved in Firmicutes exopolysaccharide production. Also described are mutations to those genes and corresponding mutated proteins to increase or stabilise exopolysaccharide production or prevent exopolysaccharide autodegradation, in particular over the course of a fermentation. Further described herein are fermentation methods and uses of the mutated genes and proteins, and of the microorganisms and the exopolysaccharides.

The invention is concerned with genes involved in Firmicutes exopolysaccharide production. The invention provides mutations of those genes and corresponding mutated proteins to increase or stabilise exopolysaccharide production or to prevent exopolysaccharide degradation, in particular over the course of a fermentation. The invention also provides suitable fermentation methods and uses of the mutated genes and proteins, of the microorganisms and the exopolysaccharides.

### BACKGROUND OF THE INVENTION

Microbial exopolysaccharides serve multiple protecting functions to the producing cell, for example protection against drought, toxins and abiotic stress, to entrap nutrients and extracellular enzymes, serve as adherents of cells to surfaces, and to maintain structural purposes by creating a propagation-conductive microenvironment as seen, e.g., in biofilms.

Thus, exopolysaccharides are a useful tool to improve survival and colonisation of surfaces by beneficial microorganisms, for example in the treatment of plants. Production of exopolysaccharides prevents or reduces washing off of plant beneficial microorganisms for example from leaves by rain or wind and improves colonisation of leaf, shoot and root surfaces. This is for example advantageous in the application of plant beneficial Paenibacilli, whose plant surface colonisation results in a significant plant protection against fungal pathogens and improvement of root nutrient uptake. As an example, biofilm polysaccharides of P. polymyxa A26 are capable of antagonizing Fusarium graminearum (Timmusk S, Copolovici D, Copolovici L, Teder T, Nevo E, Behers L. Paenibacillus polymyxa biofilm polysaccharides antagonise Fusarium graminearum. Sci Rep. 2019 Jan. 24; 9 (1): 662. doi: 10.1038/s41598-018-37718-w). The biofilm formation of some Paenibacillus species can effectively help them to colonize plant roots and help host plants to adapt and survive in harsh conditions.

In addition, exopolysaccharides and microorganisms producing exopolysaccharides can enhance soil fertility and improve yield consistency, for example by water absorption and by providing ammonia to the plants. In this context, exopolysaccharides can help to establish a microaerophilic environment around bacteria and therefore can enable the activity of the oxygen sensitive enzyme nitrogenase that is required for microbial fixation of atmospheric nitrogen.

Other applications of exopolysaccharide rely on their capacity to alter rheological properties of liquids, in particular their ability to increase the viscosity by concomitantly being biologically degradable. This is for example useful in subterraneous oil and gas extraction, cosmetics, food, feed and pharmaceutical preparations. As an example, exopolysaccharides are applied as scaffolds or matrices in tissue engineering, drug delivery and wound dressing (Nwodo U U, Green E, Okoh Al. Bacterial exopolysaccharides: functionality and prospects. Int J Mol Sci. 2012; 13 (11): 14002-14015. Published 2012 Oct. 30. doi: 10.3390/ijms131114002). Moreover, exopolysaccharides from Paenibacillus sp. were applied as antitumor agent, antioxidant or flocculant (He X, Li Q, Wang N, Chen S. Effects of an EPS Biosynthesis Gene Cluster of Paenibacillus polymyxa WLY78 on Biofilm Formation and Nitrogen Fixation under Aerobic Conditions. Microorganisms. 2021 Jan. 30; 9 (2): 289. doi: 10.3390/microorganisms9020289) indicating the wide range of biotechnological and industrial application areas for microbial exopolysaccharides. Exopolysaccharides are built from sugar units and can either consist of only one monomer (e.g. fructose in the polysaccharide levan) or different monomers. Examples for polysaccharides are, in particular but not limited thereto, glucan, fructan, curdlan, gellan, xanthan, emulsan, dextran, cellulose, aliginate, colonic acid, curdlan, dextran, diutan, levan, succinoglycan, welan and combinations thereof. Correspondingly, manufacturing materials and methods for exopolysaccharide production have been described: Publications WO2015118516 and WO2016044768 describe soil inoculation using exopolysaccharides, particularly for improving soil fertility. Publication WO2020163251 describes applications of exopolysaccharides to improve yield consistency. Publication WO2014176061 describes applications of exopolysaccharides for the treatment of subterraneous formations, in particular in oil and gas extraction. And publication WO2014160350 describes treatment of wastewater using exopolysaccharides. Materials and methods for exopolysaccharide precipitation and entrapment of plant beneficial microorganisms therein are described in WO2017151742. The genes and metabolism involved in Paenibacillus polymyxa exopolysaccharide production have been investigated in detail in Rütering et al., Tailor-made exopolysaccharides-CRISPR-Cas9 mediated genome editing in Paenibacillus polymyxa, Synthetic Biology 2017, doi: 10.1093/synbio/ysx007, which is incorporated herein in its entirety, in particular as a reference to the exopolysaccharide sugar monomer composition of Paenibacillus polymyxa and the genes involved in exopolysaccharide synthesis.

Those benefits aside exopolysaccharides are a major cause of concern in industrial liquid phase fermentations. Because of the increase in fermentation medium viscosity caused by exopolysaccharides, oxygen and nutrient transfer rates can be reduced, thereby reducing fermentation yields and requiring a higher energy for stirring of the fermentation medium. Thus, typically attempts are made to reduce or abolish exopolysaccharide production in microorganisms.

Many microorganisms produce of exopolysaccharides predominantly in log-phase growth during a fermentation. At later fermentation stages, in particular at the end of log-phase growth, exopolysaccharides are degraded and consumed as nutrients, thereby recovering some of the metabolic energy expended on exopolysaccharide production. Thus, the maximal exopolysaccharide content of a fermentation broth frequently precedes the maximum content of a target fermentation product. For example, in Paenibacillus fermentations the maximum viscosity and thus maximum exopolysaccharide content is reached before the maximum content of Fusaricidin A, B or D is reached, thereby requiring to trade exopolysaccharide content for Fusaricidin content. This is clearly undesirable particularly in plant protection applications. Furthermore, during storage of the fermentation broth after harvesting, exopolysaccharides were often degraded from cells or remaining enzymes within a few hours or days. Therefore, downstream-processing is often time-critical in order to obtain high levels of exopolysaccharides.

It was thus the object of the present invention to alleviate or remedy the aforementioned drawbacks of the prior art. In particular it was an object of the invention to provide altered nucleic acids comprising alleles which allow corresponding microorganisms to yield a higher exopolysaccharide content in the fermentation broth compared to the corresponding wild type. Furthermore, it was an object of the invention to provide such nucleic acids, alleles and microorganisms which allow to increase, compared to the corresponding wild type, the exopolysaccharide content of a fermenter content in late fermentation phases, preferably when the maximum content of at least one target fermentation product in addition to the exopolysaccharides, preferably an antimicrobial substance, produced by the microorganism is available.

### SUMMARY OF THE INVENTION

The invention provides a microorganism comprising a mutant degU gene and/or a mutant degS gene, and optionally further a mutant spoOA gene, wherein the microorganism exhibits increased and/or stabilised exopolysaccharide production and/or reduced exopolysaccharide degradation.

The invention also provides a method of increasing or stabilising exopolysaccharide production or of reduction or prevention of exopolysaccharide degradation of a microorganism, comprising the step of providing, in the microorganism, one or more of
a) a mutant degU gene, wherein
   the degU gene codes for a DegU protein having reduced DNA binding activity and/or lacks a functional DNA binding domain, and/or
   the degU gene codes for a DegU protein, wherein the mutation comprises or consists of, in decreasing order of preference for each alternative aa) and ab), one or more of:
aa) Q218*, Q218K, Q218N, Q218D, Q218R, and/or
ab) D223*, D223*+M220N, D223*+M220N+E221G, D223*+M220N+V222G, D223*+M220N+E221G+V222G, D223*+M220D, D223*+M220E, D223*+M220H, D223*+M220F, D223*+M220W, D223*+M220S, D223*+M220A;
b) a mutant degS gene, wherein
   the degS gene codes for a DegS protein lacking a functional single binding domain, a functional phosphoacceptor domain and/or a functional ATPase domain and/or
   the degS gene codes for a DegS protein, wherein the mutation comprises or consists of L99F, L99C, L99D, L99E, L99G, L99H, L99K, L99N, L99P, L99Q, L99R, L99S, L99W or L99Y;
c) a mutant spoOA gene, wherein
ca) the mutation is located in the DNA binding or receiver domain and results in a reduction or elimination of phosphorylation of the Spo0A protein and/or a reduction or elimination of dimerisation, and/or
cb) the mutation consists of or comprises any of
   A257V, more preferably A257S,
   I161R, more preferably I161L,
   in decreasing order of preference: A257S+I161I, A257A+I161L, A257V+I161I, A257S+I161F or A257A+I161R.

The invention also provides an expression vector, comprising an expression cassette for expression one or more of
a) a mutant degU gene, wherein
   the degU gene codes for a DegU protein having reduced DNA binding activity and/or lacks a functional DNA binding domain, and/or
   the degU gene codes for a DegU protein, wherein the mutation comprises or consists of, in decreasing order of preference for each alternative aa) and ab), one or more of:
aa) Q218*, Q218K, Q218N, Q218D, Q218R, and/or
ab) D223*, D223*+M220N, D223*+M220N+E221G, D223*+M220N+V222G, D223*+M220N+E221G+V222G, D223*+M220D, D223*+M220E, D223*+M220H, D223*+M220F, D223*+M220W, D223*+M220S, D223*+M220A;
b) a mutant degS gene, wherein
   the degS gene codes for a DegS protein lacking a functional single binding domain, a functional phosphoacceptor domain and/or a functional ATPase domain and/or
   the degS gene codes for a DegS protein, wherein the mutation comprises or consists of L99F, L99C, L99D, L99E, L99G, L99H, L99K, L99N, L99P, L99Q, L99R, L99S, L99W or L99Y;
c) a mutant spoOA gene, wherein
ca) the mutation is located in the DNA binding or receiver domain and results in a reduction or elimination of phosphorylation of the Spo0A protein and/or a reduction or elimination of dimerisation, and/or
cb) the mutation consists of or comprises any of
   A257V, more preferably A257S,
   I161R, more preferably I161L,
   in decreasing order of preference: A257S+I161I, A257A+I161L, A257V+I161I, A257S+I161F or A257A+I161R.

Furthermore, the invention provides a method of plant health improvement, comprising application of a microorganism comprising a mutant degU gene and/or a mutant degS gene, and optionally further a mutant spoOA gene, wherein the microorganism exhibits increased and/or stabilised exopolysaccharide production and/or reduced exopolysaccharide degradation, to
a) plant material and/or
b) a plant cultivation substrate.

And the invention provides a method of exopolysaccharide production, comprising
i) growing a microorganism comprising a mutant degU gene and/or a mutant degS gene, and optionally further a mutant spoOA gene, wherein the microorganism exhibits increased and/or stabilised exopolysaccharide production and/or reduced exopolysaccharide degradation and
ii) optionally separating the microorganism from the exopolysaccharide.

The invention also provides a use of
a microorganism comprising a mutant degU gene and/or a mutant degS gene, and optionally further a mutant spoOA gene, wherein the microorganism exhibits increased and/or stabilised exopolysaccharide production and/or reduced exopolysaccharide degradation
or of a degU gene or protein, wherein
   the degU gene codes for a DegU protein having reduced DNA binding activity and/or lacks a functional DNA binding domain, and/or
   the degU gene codes for a DegU protein, wherein the mutation comprises or consists of, in decreasing order of preference for each alternative aa) and ab), one or more of:
aa) Q218*, Q218K, Q218N, Q218D, Q218R, and/or
ab) D223*, D223*+M220N, D223*+M220N+E221G, D223*+M220N+V222G, D223*+M220N+E221G+V222G, D223*+M220D, D223*+M220E, D223*+M220H, D223*+M220F, D223*+M220W, D223*+M220S, D223*+M220A,
and/or a degS gene or DegS protein, wherein
   the degS gene codes for a DegS protein lacking a functional single binding domain, a functional phosphoacceptor domain and/or a functional ATPase domain and/or
   the degS gene codes for a DegS protein, wherein the mutation comprises or consists of L99F, L99C, L99D, L99E, L99G, L99H, L99K, L99N, L99P, L99Q, L99R, L99S, L99W or L99Y,
and/or a spoOA gene or Spo0A protein, wherein
ca) the mutation is located in the DNA binding or receiver domain and results in a reduction or elimination of phosphorylation of the Spo0A protein and/or a reduction or elimination of dimerisation, and/or
cb) the mutation consists of or comprises any of
   A257V, more preferably A257S,
   I161R, more preferably I161L,
   in decreasing order of preference: A257S+I161I, A257A+I161L, A257V+I161I, A257S+I161F or A257A+I161R
for any of:
   production of an exopolysaccharide composition,
   treatment of plants, plant leaves, plant roots and/or plant seed,
   inoculation of soil, preferably for enhancing soil fertility,
   improvement of yield consistency,
   treatment of subterraneous formations,
   treatment of wastewater,
   preparation of a pharmaceutical or a cosmetic carrier,
   preparation of pharmaceutical or cosmetic composition,
   preparation of a skin hydration composition,
   preparation of a food or feed additive
   preparation of an antitumor agent
   preparation of an antioxidant,
   preparation of a flocculant.

And the invention provides the use of one or more of
a) a mutant degU gene, wherein
   the degU gene codes for a DegU protein having reduced DNA binding activity and/or lacks a functional DNA binding domain, and/or
   the degU gene codes for a DegU protein, wherein the mutation comprises or consists of, in decreasing order of preference for each alternative aa) and ab), one or more of:
aa) Q218*, Q218K, Q218N, Q218D, Q218R, and/or
ab) D223*, D223*+M220N, D223*+M220N+E221G, D223*+M220N+V222G, D223*+M220N+E221G+V222G, D223*+M220D, D223*+M220E, D223*+M220H, D223*+M220F, D223*+M220W, D223*+M220S, D223*+M220A;
b) a mutant degS gene, wherein
   the degS gene codes for a DegS protein lacking a functional single binding domain, a functional phospoacceptor domain and/or a functional ATPase domain and/or
   the degS gene codes for a DegS protein, wherein the mutation comprises or consists of L99F, L99C, L99D, L99E, L99G, L99H, L99K, L99N, L99P, L99Q, L99R, L99S, L99W or L99Y;
c) a mutant spo0A gene, wherein
ca) the mutation is located in the DNA binding or receiver domain and results in a reduction or elimination of phosphorylation of the Spo0A protein and/or a reduction or elimination of dimerisation, and/or
cb) the mutation consists of or comprises any of
   A257V, more preferably A257S,
   I161R, more preferably I161L,
   in decreasing order of preference: A257S+I161I, A257A+I161L, A257V+I161I, A257S+I161F or A257A+I161R,
   for increasing or stabilising of exopolysaccharide production or prevention of exopolysaccharide degradation of a microorganism selected from any of the taxonomic ranks of phylum Firmicutes, class Bacilli, Clostridia or Negativicutes.

### BRIEF DESCRIPTION OF THE DRAWINGS

FIG. 1 shows the development of fermentation broth viscosity, measured according to example 3, wherein the fermentation broth was obtained from wild type Paenibacillus polymyxa DSM365 and mutants thereof during fermentations in 211 bioreactors as described in example 2. In the graphs, the viscosity profile of wild type DSM365 was always kept as a reference for comparison with the respective mutant strain. For the wild type strain, viscosity reaches a maximum of approximately 120 [mPa·s] at 100/s at 13h after start of fermentation and decreases thereafter to approximately 50 [mPa·s] at 100/s at 24 h after start of fermentation; thereafter viscosity does not change significantly. Fermentation broths of both DSM365 DegU mutant strains (strain DegU Q218* and strain DegU D223*+M220N+E221G+V222G) reach a respective viscosity of approximately 120 [mPa·s] at 100/s at 16h and 24 h after start of fermentation, respectively, and increase in viscosity to of approximately 160 [mPa·s] at 100/s at 36h and 28h, respectively. Both broth viscosities remain at or above 120 [mPa·s] at 100/s thereafter. Fermentation broth viscosity for the DegS L99F-mutant of DSM365 reaches approximately 140 [mPa·s] at 100/s at 12h after start of fermentation, peaks at approximately 200 [mPa·s] at 100/s and remains above approximately 130 [mPa·s] at 100/s. A DSM365 strain with both mutated degU Q218* and degS L99F reached approximately 120 [mPa·s] at 100/s at approximately 14h after start of fermentation and continued to increase fermentation broth viscosity to approximately 180 [mPa·s] at 100/s at 28h after start of viscosity. A strain of DSM365 having only the spo0A A257V mutation reached a fermentation broth viscosity of approximately 120 [mPa·s] at 100/s at 22h after fermentatiion start and increased fermentation broth viscosity to a peak of 140 [mPa·s] at 100/s at 32h after fermentation start. The triple mutant strain DSM365 degU Q218*+degS L99F+spo0A A257V reached approximately 120 [mPa·s] at 100/s fermentation broth viscosity at 16h after fermentation start, a peak fermentation broth viscosity of approximately 180 [mPa·s] at 100/s and continued to have a fermentation broth viscosity of at least approximately 150 [mPa·s] at 100/s thereafter.

FIG. 2 exemplarily shows a comparison of carbon transfer rate (CTR, measured by a mass spectrometer) and broth viscosity (measured according to example 3, same data as in FIG. 1) of the wildtype strain Paenibacillus DSM365.

FIG. 3 (regarding the DegS protein) shows a sequence alignment of SEQ ID NO. 2 and the sequence according to Uniprot entry A0A074LBY4_PAEPO. Numbers are given according to the position of Uniprot entry A0A074LBY4_PAEPO sequence. The number of asterisks above each amino acid of the A0A074LBY4_PAEPO sequence indicates the degree of conservation, wherein higher number of stars indicate a stronger conservation. Amino acids given below each amino acid of SEQ ID NO. 2 indicate potential substitutions allowable at the respective position, wherein “-” indicates a gap (deletion relative to the A0A074LBY4_PAEPO sequence). The possible substitutions are listed in the order of their respective preference, wherein a more preferred substitution is indicated closer to the respective position in SEQ ID NO. 2.

FIG. 4 (regarding the DegU protein) shows a sequence alignment of SEQ ID NO. 1 and the sequence according to Uniprot entry E3EBP5_PAEPS. Numbers are given according to the position of Uniprot entry E3EBP5_PAEPS sequence. The number of asterisks above each amino acid of the E3EBP5_PAEPS sequence indicates the degree of conservation, wherein higher number of stars indicate a stronger conservation. Amino acids given below each amino acid of SEQ ID NO. 1 indicate potential substitutions allowable at the respective position, wherein “-” indicates a gap (deletion relative to the E3EBP5_PAEPS sequence). The possible substitutions are listed in the order of their respective preference, wherein a more preferred substitution is indicated closer to the respective position in SEQ ID NO. 1.

FIG. 5 (regarding the Spo0A protein) shows a sequence alignment of SEQ ID NO. 3 and the sequence according to Uniprot entry A0A074LZY6_PAEPO. Numbers are given according to the position of Uniprot entry A0A074LZY6_PAEPO sequence. The number of asterisks above each amino acid of the A0A074LZY6_PAEPO sequence indicates the degree of conservation, wherein higher number of stars indicate a stronger conservation. Amino acids given below each amino acid of SEQ ID NO. 3 indicate potential substitutions allowable at the respective position, wherein “-” indicates a gap (deletion relative to the A0A074LZY6_PAEPO sequence). The possible substitutions are listed in the order of their respective preference, wherein a more preferred substitution is indicated closer to the respective position in SEQ ID NO. 3.

### BRIEF DESCRIPTION OF THE SEQUENCES

| SEQ ID | |
|---|---|
| NO. | description |
| 1 | hypothetical DegU protein screening sequence |
| 2 | hypothetical DegS protein screening sequence |
| 3 | hypothetical Spo0A protein screening sequence |
| 4 | artificial sequence for creating a degU Q218* mutation by |
| | changing nucleotide position 652 C to T |
| 5 | artificial sequence for creating a degU “M220N” (i.e. |
| | DegU D223* + M220N + E221G + V222G) mutation by |
| | inserting an A at nucleotide pos. 658 |
| 6 | artificial sequence for creating a degS L99F mutation by |
| | changing nucleotide pos. 295 C to T |
| 7 | artificial sequence for creating a spo0A A257V mutation |
| | by changing nucleotide pos. 770 C to T |
| 8 | nucleotide sequence DSM365 spo0A wild type |
| 9 | nucleotide sequence DSM365 spo0A A257V, pos. 770 C to T |
| 10 | nucleotide sequence DSM365 degU wild type |
| 11 | nucleotide sequence DSM365 degU D223* + M220N + |
| | E221G + V222G, pos. 658 A to AA |
| 12 | nucleotide sequence DSM365 degU Q218*, pos. 652 C to T |
| 13 | nucleotide sequence DSM365 degS wild type |
| 14 | nucleotide sequence DSM365 degS L99F, pos. 295 C to T |

### DETAILED DESCRIPTION OF THE INVENTION

The technical teaching of the invention is expressed herein using the means of language, in particular by use of scientific and technical terms. However, the skilled person understands that the means of language, detailed and precise as they may be, can only approximate the full content of the technical teaching, if only because there are multiple ways of expressing a teaching, each necessarily failing to completely express all conceptual connections, as each expression necessarily must come to an end. With this in mind, the skilled person understands that the subject matter of the invention is the sum of the individual technical concepts signified herein or expressed, necessarily in a pars-pro-toto way, by the innate constrains of a written description. In particular, the skilled person will understand that the signification of individual technical concepts is done herein as an abbreviation of spelling out each possible combination of concepts as far as technically sensible, such that for example the disclosure of three concepts or embodiments A, B and C are a shorthand notation of the concepts A+B, A+C, B+C, A+B+C. In particular, fallback positions for features are described herein in terms of lists of converging alternatives or instantiations. Unless stated otherwise, the invention described herein comprises any combination of such alternatives. The choice of more or less preferred elements from such lists is part of the invention and is due to the skilled person's preference for a minimum degree of realization of the advantage or advantages conveyed by the respective features. Such multiple combined instantiations represent the adequately preferred form(s) of the invention.

In so far as references are made herein to databases entries, e.g., Uniprot entries, the entries are those as published on 2021 May 1 10:00 CET. This also applies to sequences published under the corresponding database entry identifiers.

Nucleic acids and amino acids are abbreviated using their standard one- or three-letter abbreviations. Deletions are indicated by “-”, truncations are indicated by “*”. Alterations of amino acids are specified by the position of the alteration in a respective parent sequence.

As used herein, terms in the singular and the singular forms like “a”, “an” and “the” include plural referents unless the content clearly dictates otherwise. Thus, for example, use of the term “a nucleic acid” optionally includes, as a practical matter, many copies of that nucleic acid molecule; similarly, the term “probe” optionally (and typically) encompasses many similar or identical probe molecules. Also as used herein, the word “comprising” or variations such as “comprises” or “comprising” will be understood to imply the inclusion of a stated element, integer or step, or group of elements, integers or steps, but not the exclusion of any other element, integer or step, or group of elements, integers or steps.

As used herein, the term “and/or” refers to and encompasses any and all possible combinations of one or more of the associated listed items, as well as the lack of combinations when interpreted in the alternative (“or”). The term “comprising” also encompasses the term “consisting of”.

The term “about”, when used in reference to a measurable value, for example an amount of mass, dose, time, temperature, sequence identity and the like, refers to a variation of ±0.1%, 0.25%, 0.5%, 0.75%, 1%, 2%, 3%, 4%, 5%, 6%, 7%, 8%, 9%, 10%, 15% or even 20% of the specified value as well as the specified value. Thus, if a given composition is described as comprising “about 50% X,” it is to be understood that, in some embodiments, the composition comprises 50% X whilst in other embodiments it may comprise anywhere from 40% to 60% X (i.e., 50%±10%).

As used herein, the term “gene” refers to a biochemical information which, when materialised in a nucleic acid, can be transcribed into a gene product, i.e., a further nucleic acid, preferably an RNA, and preferably also can be translated into a peptide or polypeptide. The term is thus also used to indicate the section of a nucleic acid resembling said information and to the sequence of such nucleic acid (herein also termed “gene sequence”).

Also as used herein, the term “allele” refers to a variation of a gene characterized by one or more specific differences in the gene sequence compared to the wild type gene sequence, regardless of the presence of other sequence differences. Alleles or nucleotide sequence variants of the invention have at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% nucleotide “sequence identity” to the nucleotide sequence of the wild type gene. Correspondingly, where an “allele” refers to the biochemical information for expressing a peptide or polypeptide, the respective nucleic acid sequence of the allele has at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% amino acid “sequence identity” to the respective wild type peptide or polypeptide.

Mutations or alterations of amino or nucleic acid sequences can be any of substitutions, deletions or insertions; the terms “mutations” or “alterations” also encompass any combination of these. Hereinafter, all three specific ways of mutating are described in more detail by way of reference to amino acid sequence mutations; the corresponding teaching applies to nucleic acid sequences such that “amino acid” is replaced by “nucleotide”. Mutations can be introduced into the nucleotide sequence of a gene by random or directed mutagenesis techniques. Random mutagenesis techniques include for example UV irradiation and exposition to chemicals, e.g. EMS. Directed mutagenesis techniques include primer extension, meganucleases, zinc finger nucleases and CRISPR-type template directed mutagenesis.

“Substitutions” are described by providing the original amino acid followed by the number of the position within the amino acid sequence, followed by the substituted amino acid. For example, the substitution of histidine at position 120 with alanine is designated as “His120Ala” or “H120A”.

“Deletions” are described by providing the original amino acid followed by the number of the position within the amino acid sequence, followed by “−”. Accordingly, the deletion of glycine at position 150 is designated as ““Glyl50-” or “G150-”. Alternatively, deletions are indicated by e.g. “deletion of D183 and G184”.

“Terminations” are described by providing the original amino acid followed by the number of the position within the amino acid sequence, followed by “*”. Accordingly, an amino acid chain termination at position 150 instead of a glycine at this position is designated as “Glyl50*” of “G150*”.

“Insertions” are described by providing the original amino acid followed by the number of the position within the amino acid sequence, followed by the original amino acid and the additional amino acid. For example, an insertion at position 180 of lysine next to glycine would be designated as “Glyl80GlyLys” or “G180GK”. When more than one amino acid residue is inserted, such as e.g. a Lys and Ala after Glyl80 this may be indicated as: Glyl80GlyLysAla or G180GKA. In cases where a substitution and an insertion occur at the same position, this may be indicated as S99SD+S99A or in short S99AD. In cases where an amino acid residue identical to the existing amino acid residue is inserted, it is clear that degeneracy in the nomenclature arises. If for example a glycine is inserted after the glycine in the above example this would be indicated by G180GG.

Variants comprising multiple alterations are separated by “+”, e.g., “Arg170Tyr+Glyl95Glu” or “R170Y+G195E” representing a substitution of arginine and glycine at positions 170 and 195 with tyrosine and glutamic acid, respectively. Alternatively, multiple alterations may be separated by space or a comma, e.g., R170Y G195E or R170Y, G195E respectively.

Where different alterations can be introduced at a position, the different alterations are separated by a comma, e.g., “Arg170Tyr, Glu” represents a substitution of arginine at position 170 with tyrosine or glutamic acid. Alternatively, different alterations or optional substitutions may be indicated in brackets e.g. Arg170 [Tyr, Gly] or Arg170 {Tyr, Gly} or in short R170 [Y,G] or R170 {Y, G}.

A special aspect concerning amino acid substitutions are conservative mutations which often appear to have a minimal effect on protein folding resulting in substantially maintained peptide or polypeptide properties of the respective peptide or polypeptide variant compared to the peptide or polypeptide properties of the parent peptide or polypeptide. Conservative mutations are those where one amino acid is exchanged with a similar amino acid. For determination of %-similarity the following applies, which is also in accordance with the BLOSUM62 matrix, which is one of the most used amino acids similarity matrix for database searching and sequence alignments:
Amino acid A is similar to amino acids S
Amino acid D is similar to amino acids E, N
Amino acid E is similar to amino acids D, K and Q
Amino acid F is similar to amino acids W, Y
Amino acid H is similar to amino acids N, Y
Amino acid I is similar to amino acids L, M and V
Amino acid K is similar to amino acids E, Q and R
Amino acid L is similar to amino acids I, M and V
Amino acid M is similar to amino acids I, L and V
Amino acid N is similar to amino acids D, H and S
Amino acid Q is similar to amino acids E, K and R
Amino acid R is similar to amino acids K and Q
Amino acid S is similar to amino acids A, N and T
Amino acid T is similar to amino acids S
Amino acid V is similar to amino acids I, L and M
Amino acid W is similar to amino acids F and Y
Amino acid Y is similar to amino acids F, H and W

Conservative amino acid substitutions may occur over the full length of the sequence of a polypeptide sequence of a functional protein such as a peptide or polypeptide. Preferably such mutations are not pertaining the functional domains of a peptide or polypeptide.

Protein or nucleic acid variants may be defined by their sequence identity when compared to a parent protein or nucleic acid. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

Seq A: AAGATACTG length: 9 bases
Seq B: GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “|” symbol in the alignment indicates identical residues (which means bases for DNA or amino acids for proteins). The number of identical residues is 6.

The “-” symbol in the alignment indicates gaps. The number of gaps introduced by alignment within the sequence B is 1. The number of gaps introduced by alignment at borders of sequence B is 2, and at borders of sequence A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

Producing a pairwise alignment which is showing sequence B over its complete length according to the invention consequently results in:

Seq A: GATACTG-
||| |||
Seq B: GAT-CTGA

The alignment length showing the shorter sequence over its complete length is 8 (one gap is present which is factored in the alignment length of the shorter sequence).

Accordingly, the alignment length showing sequence A over its complete length would be 9 (meaning sequence A is the sequence of the invention), the alignment length showing sequence B over its complete length would be 8 (meaning sequence B is the sequence of the invention).

After aligning the two sequences, in a second step, an identity value shall be determined from the alignment. Therefore, according to the present description the following calculation of percent-identity applies:
%-identity=(identical residues/length of the alignment region which is showing the respective sequence of this invention over its complete length)*100. Thus, sequence identity in relation to comparison of two amino acid sequences according to the invention is calculated by dividing the number of identical residues by the length of the alignment region which is showing the respective sequence of this invention over its complete length. This value is multiplied with 100 to give “%-identity”. According to the example provided above, %-identity is: for sequence A being the sequence of the invention (6/9)* 100=66.7%; for sequence B being the sequence of the invention (6/8)*100=75%.

The term “expression cassette” means those constructs in which the nucleic acid sequence encoding an amino acid sequence to be expressed is linked operably to at least one genetic control element which enables or regulates its expression (i.e. transcription and/or translation). The expression may be, for example, stable or transient, constitutive or inducible. Ex-pression cassettes may also comprise the coding regions for two or more polypeptides and lead to the transcription of polycistronic RNAs.

The terms “express,” “expressing,” “expressed” and “expression” refer to expression of a gene product (e.g., a biosynthetic enzyme of a gene of a pathway or reaction defined and described in this application) at a level that the resulting enzyme activity of this protein encoded for, or the pathway or reaction that it refers to allows metabolic flux through this pathway or reaction in the organism in which this gene/pathway is expressed in. The expression can be done by genetic alteration of the microorganism that is used as a starting organism. In some embodiments, a microorganism can be genetically altered (e.g., genetically engineered) to express a gene product at an increased level relative to that produced by the starting microorganism or in a comparable microorganism which has not been altered. Genetic alteration includes, but is not limited to, altering or modifying regulatory sequences or sites associated with expression of a particular gene (e.g. by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for exam-pie, to block expression of repressor proteins).

The terms “overexpress”, “overexpressing”, “overexpressed” and “overexpression” refer to expression of a gene product, in particular to enhancing the expression of a gene product at a level greater than that present prior to a genetic alteration of the starting microorganism. In some embodiments, a microorganism can be genetically altered (e.g., genetically engineered) to express a gene product at an increased level relative to that produced by the starting microorganism. Genetic alteration includes, but is not limited to, altering or modifying regulatory sequences or sites associated with expression of a particular gene (e.g., by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for example, to block expression of repressor proteins). Another way to overexpress a gene product is to enhance the stability of the gene product to increase its life time. The terms “overexpress”, “overexpressing”, “overexpressed” and “overexpression” can also mean that a gene activity is introduced into a microorganism where the respective gene activity, has not been observed before, e.g. by introducing a recombinant gene, e.g. a heterologous gene, in one or more copies into the microorganism preferably by means of genetic engineering.

The term “plant” is used herein in its broadest sense as it pertains to organic material and is intended to encompass eukaryotic organisms that are members of the taxonomic kingdom plantae, examples of which include but are not limited to monocotyledon and dicotyledon plants, vascular plants, vegetables, grains, flowers, trees, herbs, bushes, grasses, vines, ferns, mosses, fungi and algae, etc, as well as clones, offsets, and parts of plants used for asexual propagation (e.g. cuttings, pipings, shoots, rhizomes, underground stems, clumps, crowns, bulbs, corms, tubers, rhizomes, plants/tissues produced in tissue culture, etc.). Unless stated otherwise, the term “plant” refers to a whole plant, any part thereof, or a cell or tissue culture derived from a plant, comprising any of: whole plants, plant components or organs (e.g., leaves, stems, roots, etc.), plant tissues, seeds, plant cells, and/or progeny of the same. A plant cell is a biological cell in a plant or plant part, taken from a plant or derived through culture from a cell taken from a plant.

Plants that are particularly useful for the purposes of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp., Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. According to a preferred embodiment of the present invention, the plant is a crop plant. Examples of crop plants include inter alia soybean, sunflower, canola, alfalfa, rapeseed, cotton, tomato, potato, maize or tobacco.

According to the invention, a plant is cultivated to yield plant material. Cultivation conditions are chosen in view of the plant and may include, for example, any of growth in a greenhouse, growth on a field, growth in hydroculture and hydroponic growth. Plants and plant parts, for example seeds and cells, can be genetically modified. In particular, plants and parts thereof, preferably seed and cells, can be recombinant, preferably transgenic or cisgenic.

The term “plant material” denotes any tissue, organ or material produced by a plant, including, but are not limited to, plant cells, stems, roots, flowers, plant propagation material, ovules, stamens, seeds, leaves, embryos, meristematic regions, callus tissue, anther cultures, gametophytes, sporophytes, pollen, microspores, protoplasts, hairy root cultures, straw, husks, fruit and nut shells. As used herein, a “plant cell” includes, but is not limited to, a protoplast, gamete producing cell, and a cell that regenerates into a whole plant. The term “plant propagation material” is to be understood to denote all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion in or pouring of the plant health promotion composition of the present invention.

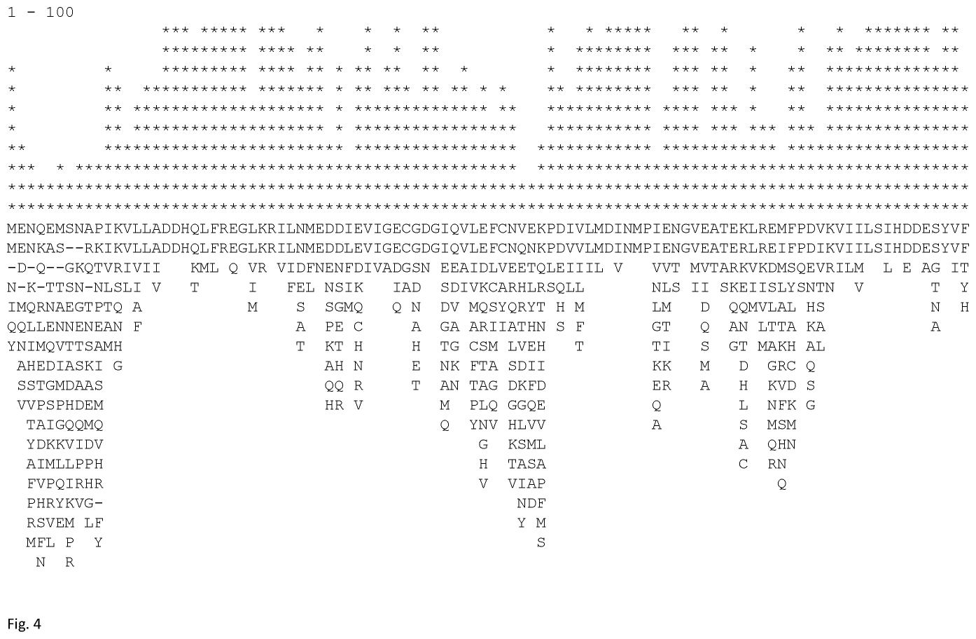

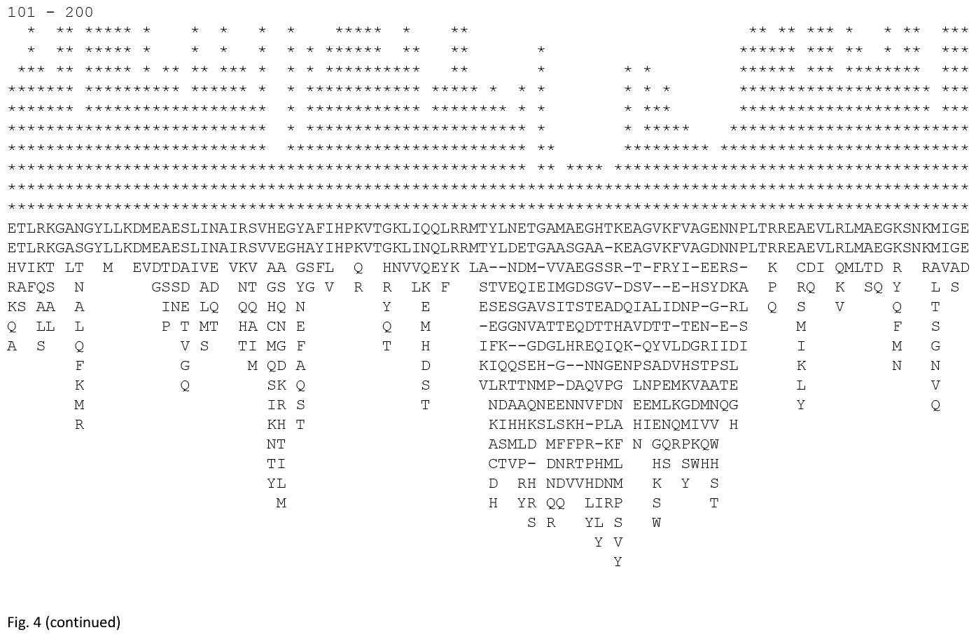

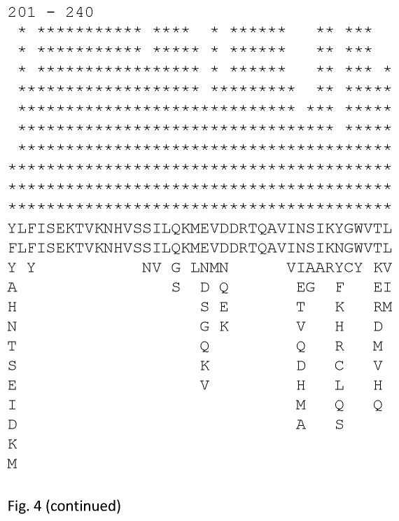

The invention provides a microorganism comprising a mutant degU gene. The mutant degU gene, when expressed in the microorganism, results in the production of a mutant DegU protein; as a degU gene codes for a DegU protein. According to the invention a wild type DegU protein is a member of the CheY-like superfamily (InterPro ID IPR011006) and comprises, using InterPro notation, a signal transduction response regulator (receiver domain) (IPR001789) and a transcription regulator LuxR domain (C-terminal) (IPR000792). According to Pfam nomenclature the wild type DegU protein comprises a response regulator receiver domain (PF00072, Pao et al., J Mol Evol 1995, 136-154 Response regulators of bacterial signal transduction systems: selective domain shuffling during evolution), and a LuxR-type DNA-binding HTH domain (PF00196). Preferably the wild type degU gene codes for a DegU protein whose amino acid sequence has at least 40%, more preferably at least 43%, more preferably at least 45%, more preferably at least 53%, more preferably at least 57%, more preferably at least 70%, more preferably at least 77%, more preferably at least 85%, more preferably at least 88% sequence identity to SEQ ID NO. 1, wherein preferably the sequence identity to SEQ ID NO. 1 is at most 95%, more preferably at most 92%. Particularly preferred the wild type DegU protein has 50-95% sequence identity to SEQ ID NO. 1, more preferably 77-91%. It is to be understood that SEQ ID NO. 1 is an artificial amino acid sequence specifically constructed as a template for amino acid sequence screening and annealing purposes. The sequence can thus be used for identification of degU genes independent from the fact that no DegU activity of the polypeptide of SEQ ID NO. 1 is shown herein. Particularly preferred as a wild type DegU gene in a method or plant according to the present invention is any of the amino acid sequences defined by the following Uniprot identifiers, in decreasing order of preference: E3EBP5_PAEPS, A0A4R6MUX9_9BACL, A0A268SA79_9BACL, A0A069DEZ2_9BACL, A0A0B0HVN5_9BACL, W4EI28_9BACL, A0A1X7GB62_9BACL, A0A089MEU3_9BACL, A0A0E4HEC8_9BACL, A0A4P8XUS1_9BACL, A0A0M2VKR6_9BACL, A0A089M364_9BACL, V9GIW8_9BACL, W7YTM0_9BACL, A0A098MFT1_9BACL, D3EMG0_GEOS4, A0A1B8VU54_9BACL, A0A2Z2KSF3_9BACL, A0A269W3P3_9BACL, A0A1R1EEL5_9BACL, X5A6E5_9BACL, A0A089IT67_9BACL, A0A110JV80_9BACL, A0A168QEL2_9BACL, A0A0D3VFM7_9BACL, A0A172ZLN3_9BACL, A0A167D848_9BACL, A0A1E3L0K1_9BACL, A0A2W1LCB1_9BACL, A0A0U2N3N5_9BACL, L0EHW2_THECK, A0A1T2X729_9BACL, A0A1B8UUC0_9BACL, H3S9W0_9BACL, A0A3D9SC72_9BACL, A0A401I4R6_9BACL, A0A116WHZ6_9BACL, A0A015NM30_9BACL, A0A0F5R725_9BACL, A0A2N5NDN0_9BACL, M9LLL0_PAEPP, A0A0D5NRR7_9BACL, A0A2S0UEL3_9BACL, A0A4Q2M117_9BACL, A0A1H1WMP7_9BACL, A0A3A1US45_9BACL, A0A3G9JII4_9BACL, C6D5A1_PAESJ, A0A433XGQ7_9BACL, A0A113PZK5_9BACL, A0A1R1DAD8_9BACL, A0A4P6F1N4_9BACL, A0A0Q4R517_9BACL, A0A172TIH8_9BACL, A0A2V4X724_9BACL, A0A1Y5KD60_9BACL, A0A368VSS2_9BACL, A0A1B8VZY5_9BACL, A0A371P0X4_9BACL, A0A231RB89_9BACL, A0A369BC27_9BACL, E0IEE4_9BACL, A0A2V2YZQ8_9BACL, A0A1G7PNR5_9BACL, A0A3S1BJF8_9BACL, A0A1A5YDL9_9BACL, A0A0U2WGN9_9BACL, A0A494X986_9BACL, A0A3D9KD00_9BACL, C6J2I4_9BACL, A0A3Q9IF25_9BACL, A0A3G3K2Z7_9BACL, A0A090XUD0_PAEMA, A0A3D91787_9BACL, A0A398CFS8_9BACL, A0A1B1N3Y9_9BACL, A0A081P316_9BACL, A0A3T1DDG1_9BACL, A0A1K1QXK1_9BACL, A0A3Q8SA76_9BACL, A0A1X7KWC6_9BACL, A0A229USY4_9BACL, A0A4Q9DKY4_9BACL, A0A4R5KE70_9BACL, A0A329L4V8_9BACL, A0A2W1N4T3_9BACL, A0A1I1BB61_9BACL, H6NT64_9BACL, A0A1I4LD00_9BACL, A0A329MBB6_9BACL, A0A3S1AKF3_9BACL, F5LST8_9BACL, A0A1V4HGJ1_9BACL, A0A1H0L0T9_9BACL, A0A0Q7JPS4_9BACL, A0A1H4RQ86_9BACL, A0A3S0BTA6_9BACL, A0A1C0ZYC4_9BACL, A0A0C2RFX7_9BACL, V9W4A0_9BACL, A0A2V5KBB6_9BACL, A0A3B0C3G8_9BACL, A0A4R4EFH3_9BACL, A0A1U9KAL0_9BACL, A0A4R3KIF8_9BACL, A0A292YJB9_9BACL, A0A075RHH4_BRELA, A0A0D1XDF4_ANEMI, A0A1A5XJS0_9BACL, V6M9Z2_9BACL, A0A120HRZ5_9BACL, A0A419V950_9BACL, A0A3R9QNM1_9BACL, A0A1I4L117_9BACL, A0A1H0J2F9_9BACI, A0A3M8DYQ5_9BACL, A0A1I2EIB9_9BACI, A0A428N9S8_9BACI, A0A2P6MHC1_9BACI, A0A1I4CG56_9BACL, C0Z730_BREBN, M8DFP6_9BACL, A0A345BZD8_9BACI, A0A419SF78_9BACL, A0A3M8BE38_9BACL, A0A1H9W953_9BACI, A0A4Q1ST01_9BACL, F5L9B2_CALTT, A0A1G8AEE7_9BACI, D6Y0E8_BACIE, A0A4Q0VW28_9BACI, A0A2T4U7P0_9BACI, A0A061NX68_9BACL, A0A061P3R3_9BACL, A0A098EIU7_9BACL, A0A3M8P3C8_9BACL, A0A1H2UAM2_9BACI, A0A3A9KCQ9_9BACI, A0A1Y0IJ22_9BACL, A0A1G8E1Q8_9BACI, A0A1S2M8P6_9BACI, Q9K6U7_BACHD, A0A4R3N1F6_9BACI, A0A437KCI7_9BACI, A0A2P8GCB9_9BACL, A0A1X9MFG9_9BACI, A0A1H9TTG1_9BACI, A0A327YHU2_9BACI and A0A368Y3Q5_9BACI. Particularly preferred according to the invention are wild type DegU protein sequences and corresponding degU genes coding therefor which have at least 45%, more preferably at least 51%, more preferably at least 54% and even more preferably 73-100% sequence identity to the amino acid sequence given by Uniprot identifier E3EBP5_PAEPS. When not considering the specific mutations to the DegU protein sequence described according to the invention, the mutant DegU protein preferably differs from the amino acid sequence given by Uniprot identifier E3EBP5_PAEPS by 0-20 amino acids, more preferably 0-15 amino acids, even more preferably 0-10 amino acids, even more preferably 1-5 amino acids, wherein those differences preferably conform to the constraints according to FIG. 4. If the mutant DegU sequence, when aligned to the sequence according to Uniprot identifier E3EBP5_PAEPS, is longer than said sequence, then each C- or N-terminal extension is preferably no longer than 10 amino acids, more preferably 0-5 amino acids.

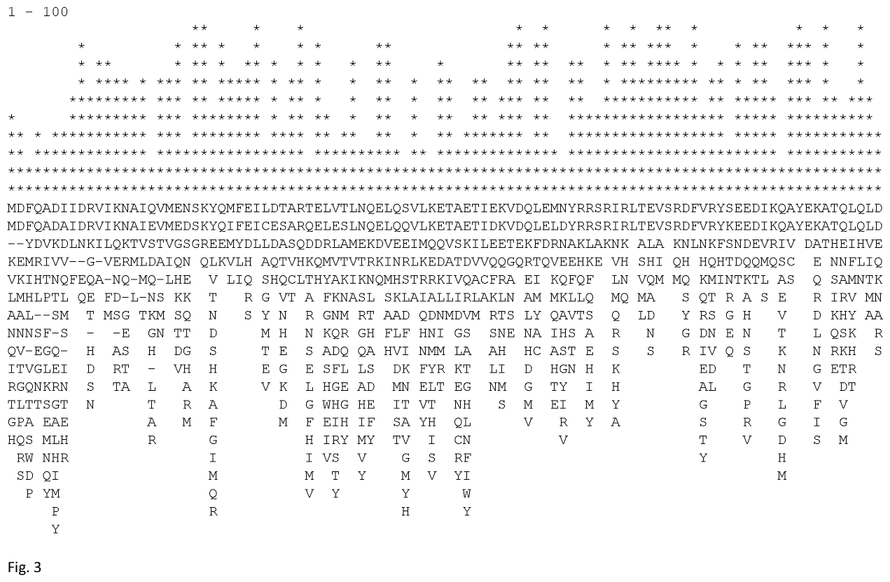

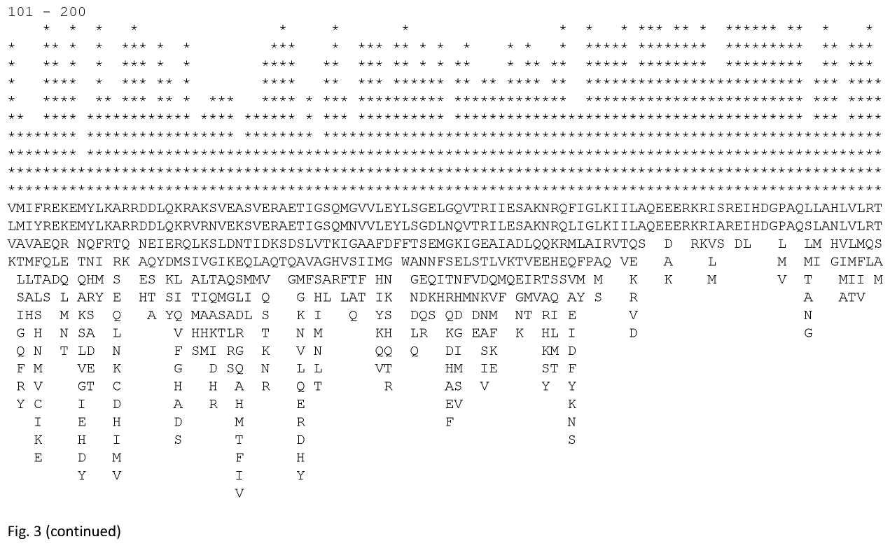

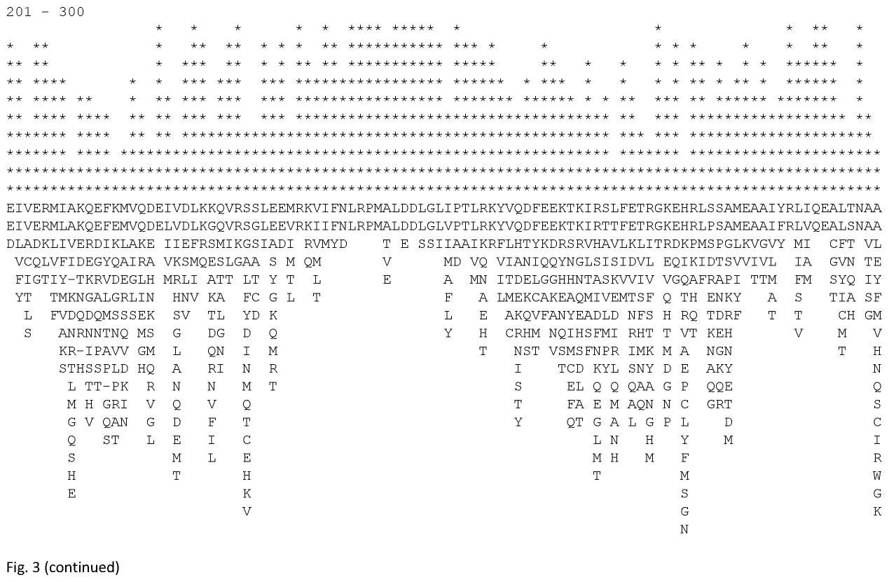

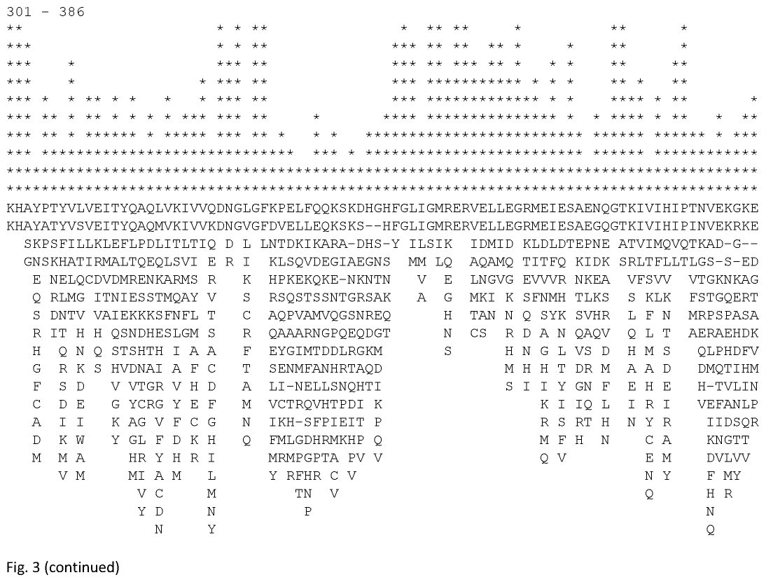

The invention also provides a microorganism comprising a mutant degS gene. The mutant degS gene, when expressed in the microorganism, results in the production of a mutant DegS protein; as a degS gene codes for a DegS protein. According to the invention a wild type DegS protein is a member of the DegS type signal transduction histidine kinase family (InterPro ID IPR016381) and comprises, using InterPro notation, a sensor DegS domain (IPR008595) and a histidine kinase domain (IPR005467). According to Pfam nomenclature the wild type DegS protein comprises a sensor protein DegS domain (PF05384), a HisKA_3 histidine kinase domain (PF07730) and a HATPase_c GHKL domain (PF02518). Preferably the wild type degS gene codes for a DegS protein whose amino acid sequence has at least 40%, more preferably at least 43%, more preferably at least 46%, more preferably at least 50%, more preferably at least 58%, more preferably at least 64%, more preferably at least 79%, more preferably at least 84% sequence identity to SEQ ID NO. 2, wherein preferably the sequence identity to SEQ ID NO. 2 is at most 95%, more preferably at most 91%. Particularly preferred the wild type DegS protein has 50-95% sequence identity to SEQ ID NO. 2, more preferably 58-89%. It is to be understood that SEQ ID NO. 2 is an artificial amino acid sequence specifically constructed as a template for amino acid sequence screening and annealing purposes. The sequence can thus be used for identification of degS genes independent from the fact that no DegS activity of the polypeptide of SEQ ID NO. 2 is shown herein. Particularly preferred as a wild type degS gene in a method or plant according to the present invention is any of the amino acid sequences defined by the following Uniprot identifiers, in decreasing order of preference: A0A074LBY4_PAEPO, E3EBP6_PAEPS, A0A4R6MVR0_9BACL, A0A069DLG2_9BACL, A0A268SAI9_9BACL, A0A1X7GB86_9BACL, A0A0M2VLZ1_9BACL, A0A1R1EED0_9BACL, A0A0B0HR83_9BACL, A0A4P8XRM7_9BACL, W7YPT3_9BACL, A0A433XGY7_9BACL, D3EMG1_GEOS4, V9GK22_9BACL, A0A269W177_9BACL, A0A3Q8SA22_9BACL, A0A1E3L2X6_9BACL, A0A369BCF2_9BACL, A0A2S0UEJ3_9BACL, A0A090XSK7_PAEMA, A0A3S1DMQ5_9BACL, A0A1B1N3X4_9BACL, C6J213_9BACL, A0A172ZLS7_9BACL, A0A1G7PLD1_9BACL, A0A2Z2KM36_9BACL, A0A3Q9IDP6_9BACL, A0A0D3VFE7_9BACL, A0A168QEJ2_9BACL, A0A1B8VU57_9BACI, W4EHN2_9BACL, A0A0E4CZI9_9BACL, A0A089L4Y0_9BACL, A0A098MEC1_9BACL, A0A089IPW0_9BACL, A0A167D837_9BACL, A0A089NAN3_9BACL, X4ZSE0_9BACL, A0A2W1M2J1_9BACL, A0A110JTZ3_9BACL, A0A4Q2LW12_9BACL, A0A113PZ40_9BACL, A0A401I4T4_9BACL, A0A1B8UU84_9BACL, A0A1T2X709_9BACL, A0A2N5NDJ2_9BACL, A0A0F5RAF5_9BACL, A0A3G9IYB3_9BACL, A0A1H1WM07_9BACL, H3S9W1_9BACL, A0A116WIJ5_9BACL, A0A0D5NQK3_9BACL, A0A015KKW1_9BACL, A0A3D9SD21_9BACL, A0A1B8VZZ2_9BACI, M9LFD8_PAEPP, A0A2V4WBE9_9BACL, A0A0U2WI25_9BACL, A0A1R1DAI7_9BACL, A0A368VS81_9BACL, E0IEE5_9BACL, A0A371P0S2_9BACL, A0A4P6EY40_9BACL, L0EJK6_THECK, A0A3D91772_9BACL, A0A1X7KY93_9BACL, A0A231R9F5_9BACL, A0A3A1UXN0_9BACL, A0A494XFI9_9BACL, A0A1Y5KD15_9BACL, A0A398CI63_9BACL, A0A3T1DDD1_9BACL, A0A0Q4RDM1_9BACL, C6D5A2_PAESJ, A0A2V2Z262_9BACL, A0A3G3K194_9BACL, A0A3D9KBV6_9BACL, A0A1A5YD71_9BACL, A0A433R8L8_9BACL, A0A172TIT6_9BACL, A0A1I1BCS1_9BACL, A0A081P315_9BACL, A0A4R5KGY1_9BACL, A0A229USJ3_9BACL, A0A2V5JWK3_9BACL, A0A329MCI0_9BACL, A0A0U2INE8_9BACL, A0A1K1QX93_9BACL, A0A2W1NWZ0_9BACL, A0A1I4LCQ8_9BACL, A0A329L6X4_9BACL, A0A0C2V9A2_9BACL, A0A4Q9DIC7_9BACL, H6NT63_9BACL, A0A1H4RQL7_9BACL, A0A3B0C2F8_9BACL, V9VZ78_9BACL, A0A1H0L1L7_9BACL, A0A430JA16_9BACL, F5LST9_9BACL, A0A4R4EAF4_9BACL, A0A0Q7JRA6_9BACL, A0A1V4HGJ0_9BACL, A0A1C0ZYJ1_9BACL, A0A4R3KJJ5_9BACI, M8DFK7_9BACL, A0A1U9KAR4_9BACL, A0A3M8DWV1_9BACL, A0A1A5XKA3_9BACL, A0A1E5LA89_9BACL, A0A074LTT3_9BACL, A0A1I4CE81_9BACL, C0Z731_BREBN, V6MBX1_9BACL, A0A1Y0IJ16_9BACL, A0A3M8BE71_9BACL, A0A4Q1STZ5_9BACL, A0A1E5G3N9_9BACL, A0A075RB77_BRELA, A0A419SF93_9BACL, A0A1Z5HTH4_9THEO, F5L9B1_CALTT, A0A3S9T1P5_9FIRM, A0A2N5M9N1_9BACI, A0A235FGA1_9BACI, A0A0M2U6G0_9FIRM, A0A4R6TU87_9BACI, A0A1E5LDM8_9BACI, A0A498RIM9_9FIRM, A0A120HRZ3_9BACL, A0A4Q0VV23_9BACI, A0A1I2EJ29_9BACI, A0A1U7MGK1_9FIRM, E6TSA5_BACCJ, A0A3E2JMS2_9BACI, A0A1I4L1V6_9BACI, A0A2P8HQR2_9BACI, Q9K6U6_BACHD, A0A402BS91_9FIRM, A0A4R3MVB0_9BACI, X0RMB4_9BACI, A0A4R2RM72_9FIRM, A0A1H4AT83_9BACI, A0A292YC86_9BACL, C5D873_GEOSW, Q5KV50_GEOKA, A0A1W2BFW9_9FIRM, A0A285CZH1_9BACI and A0A1G9QKZ2_9FIRM. Particularly preferred according to the invention are wild type DegS protein sequences and corresponding degS genes coding therefor which have at least 40%, more preferably at least 46%, more preferably at least 58% and even more preferably 80-100% sequence identity to the amino acid sequence given by Uniprot identifier A0A074LBY4_PAEPO. When not considering the specific mutations to the DegS protein sequence described according to the invention, the mutant DegS protein preferably differs from the amino acid sequence given by Uniprot identifier A0A074LBY4_PAEPO by 0-40 amino acids, more preferably 0-20 amino acids, even more preferably 0-10 amino acids, even more preferably 1-5 amino acids, wherein those differences preferably conform to the constraints according to FIG. 3. If the mutant DegS sequence, when aligned to the sequence according to Uniprot identifier A0A074LBY4_PAEPO, is longer than said sequence, then each C- or N-terminal extension is preferably no longer than 10 amino acids, more preferably 0-5 amino acids.

The microorganism according to the present invention can comprise either a mutant degU gene or a mutant degS gene, or the microorganism comprises both a mutant degU gene and a mutant degS gene. Correspondingly the microorganism according to the present invention can comprise either a mutant DegU protein or a mutant DegS protein, or the microorganism comprises both a mutant DegU and a mutant DegS protein.

According to the invention, the microorganism can comprise a mutant spoOA gene. The mutant spo0A gene, when expressed in the microorganism, results in the production of a mutant Spo0A protein; as a spoOA gene codes for a Spo0A protein. According to the invention a wild type Spo0A protein is a member of the Sporulation transcription factor Spo0A (IPR012052) and comprises, using InterPro notation, a Signal transduction response regulator receiver domain (IPR001789) and a Sporulation initiation factor Spo0A C-terminal domain (IPR014879), which is part of a Winged helix-like DNA-binding domain superfamily (IPR036388). According to Pfam nomenclature the wild type Spo0A protein comprises a Response regulator receiver domain (PF00072) and a Sporulation initiation factor Spo0A C terminal domain (PF08769). Preferably the wild type spoOA gene codes for a Spo0A protein whose amino acid sequence has at least 45%, more preferably at least 56%, more preferably at least 69%, more preferably at least 70%, more preferably at least 67%, more preferably at least 70%, more preferably at least 73%, more preferably at least 74%, more preferably 75% sequence identity to SEQ ID NO. 3, wherein preferably the sequence identity to SEQ ID NO. 3 is at most 85%, more preferably at most 11%.

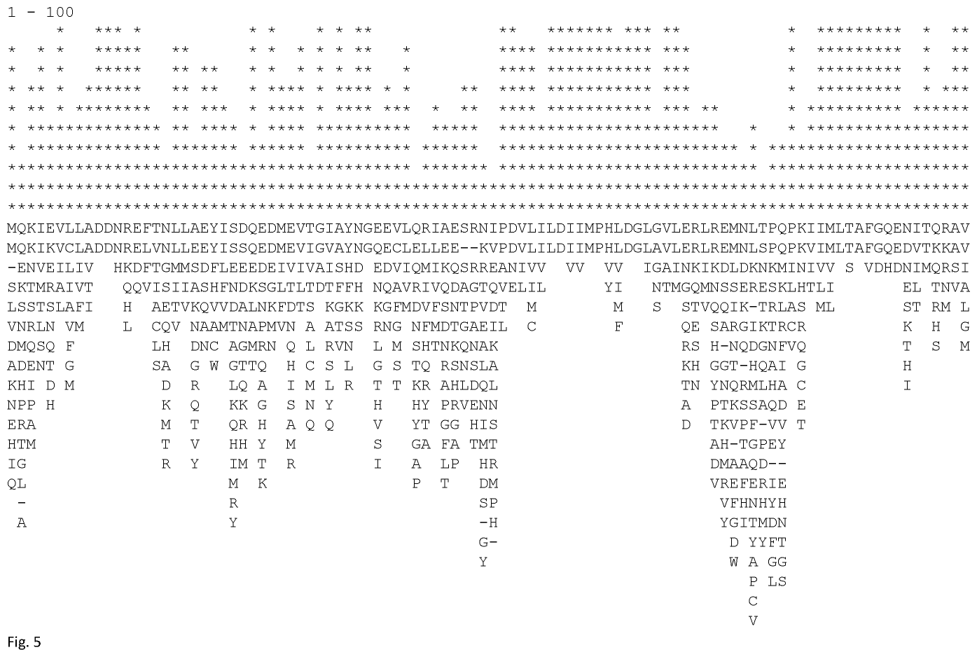

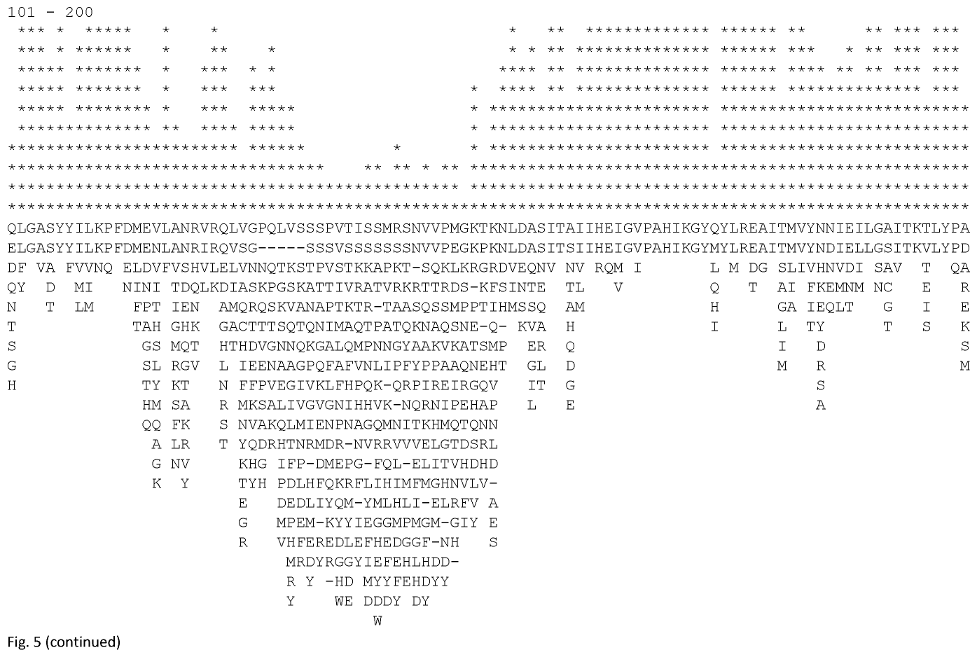

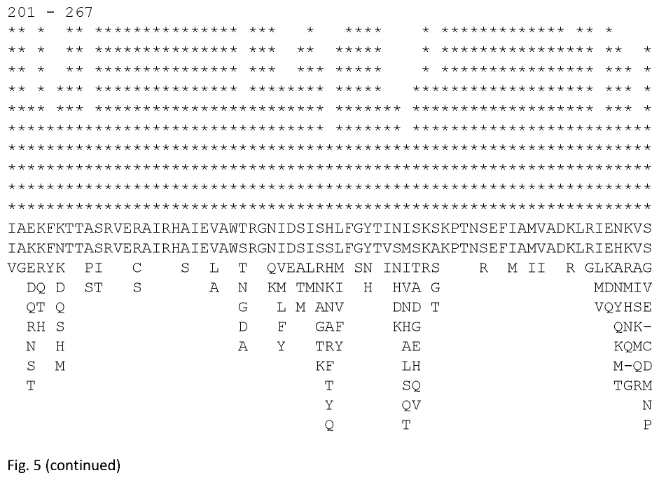

Particularly preferred the wild type Spo0A protein has 50-85% sequence identity to SEQ ID NO. 3, more preferably 76-84%. It is to be understood that SEQ ID NO. 3 is an artificial amino acid sequence specifically constructed as a template for amino acid sequence screening and annealing purposes. The sequence can thus be used for identification of spoOA genes independent from the fact that no Spo0A activity of the polypeptide of SEQ ID NO. 3 is shown herein. Particularly preferred as a wild type spo0A gene in a method or plant according to the present invention is any of the amino acid sequences defined by the following Uniprot identifiers, in decreasing order of preference: A0A074LZY6_PAEPO, E0RDX7_PAEP6, H6CM41_9BACL, A0A0D7WZ78_9BACL, A0A167DI09_9BACL, W7YKB3_9BACL, A0A168BRF7_9BACL, A0A1G5JWJ2_9BACL, A0A168P4Q5_9BACL, A0A168M3D7_9BACL, A0A1R1EUX4_9BACL, A0A2W6PE29_9BACL, A0A2V4WTN3_PAEBA, A0A328WGM0_PAELA, D3E6N2_GEOS4, G4HF05_9BACL, A0A1R0XBX0_9BACL, A0A098M8U8_9BACL, A0A3Q8SBT8_9BACL, A0A0M1P3N3_9BACL, R9LQX4_9BACL, A0A2Z2KRN4_9BACL, A0A1B8WQN2_9BACI, A0A089MEU2_9BACL, A0A089LZP7_9BACL, A0A0F7FA95_PAEDU, A0A0E4HDK7_9BACL, A0A1G7R7Q0_9BACL, A0A1H8N6P6_9BACL, X4ZFA8_9BACL, A0A3G9IQE6_9BACL, A0A369BNP1_9BACL, A0A1B1N013_9BACL, A0A015KRJ2_9BACL, A0A2N5N5F6_9BACL, A0A1T2XNU8_9BACL, A0A090ZFJ2_PAEMA, A0A3D9QX06_9BACL, E0ICH6_9BACL, A0A1G9E5Z0_9BACL, A0A3S1DUM5_9BACL, A0A0D5NPL4_9BACL, A0A368WCL4_9BACL, A0A4Q2LM98_9BACL, A0A328U1G0_9BACL, A0A172TM01_9BACL, A0A1I2EKA8_9BACL, A0A1A5YCA7_9BACL, A0A371PM84_9BACL, A0A3A6PB13_9BACL, A0A2V2YXM7_9BACL, L0EEN3_THECK, A0A3A1UXY9_9BACL, A0A3B0CH88_9BACL, A0A1V4HR00_9BACL, A0A1V0UWJ6_9BACL, H3SFG5_9BACL, A0A1X7JKH5_9BACL, A0A1I2FDS6_9BACL, A0A3D9IJB3_9BACL, A0A398CE46_9BACL, M9LB51_PAEPP, A0A3D9KSR7_9BACL, A0A081NWT7_9BACL, H6NL94_9BACL, A0A1C0ZWF8_9BACL, A0A4Y8M823_9BACL, A0A1X7HJ70_9BACL, A0A329MFB7_9BACL, A0A1G4P4T7_9BACL, A0A229UXF4_9BACL, A0A0U2WBQ5_9BACL, A0A3S0BM74_9BACL, K4ZP76_PAEA2, A0A2W1NBK6_9BACL, A0A172ZK56_9BACL, A0A3M8CIR1_9BACL, M8EE17_9BACL, A0A0Q3T5E2_BRECH, A0A0K9YRB7_9BACL, A0A113U483_9BACL, V6MCA1_9BACL, C0ZC17_BREBN, A0A4R3KM88_9BACI, A0A3M8B6E4_9BACL, A0A2N3LN87_9BACI, A0A419SMW9_9BACL, A0A3M8D088_9BACL, A0A075R4A3_BRELA, U1X7N0_ANEAE, A0A1H2UFN8_9BACL, A0A0D1VW72_ANEMI, A0A0X8D3E6_9BACL, A0A0U5AZK5_9BACL, A0A4R3L002_9BACL, A0A0Q3WXA1_9BACI, A0A0B0IAE5_9BACI, A0A223KSV6_9BACI, W4PXN5_9BACI, A0A235BCM6_9BACL, A0A235FAK4_9BACI, A0A2T4Z9J8_9BACL, A0A1S2MEZ1_9BACI, Q9K977_BACHD, A0A1S2LUZ3_9BACI, A0A1U9KC16_9BACL, A7Z6J0_BACVZ, Q65HJ7_BACLD, W4QWX1_BACA3, A0A116TUX2_9BACL, A0A1/2L311_9BACL, A0A0H3E179_BACA1, SP0A_BACSU, A8FF06_BACP2, A0A0J6EVC7_9BACI, A0A417YV34_9BACI, D5DS62_BACMQ, A0A4Q0VQU7_9BACI, A0A1H9PKN5_9BACI, A0A113QAI8_9BACL, A0A1G6Q9T8_9BACL, W1SHY1_9BACI, A0A364K8M0_9BACL, A0A150F6K4_9BACI, M5PEN8_9BACI, A0A1S2M754_9BACI, A0A0A8X8S8_9BACI, A0A1R1RU53_9BACI, A0A1S2LYV1_9BACI, A0A1B3XQX6_9BACI, A0A1H8EQX3_9BACL, A0A2N5GRE3_9BACI, A0A4R1B005_9BACI, A0A4R1QFH8_9BACI, A0A1B1Z5W5_9BACI, K6BXH2_9BACI, A0A160F753_9BACI, U5LDF9_9BACI, A0A0M0KYT7_9BACI, A0A061NL57_9BACL, A0A3A1QZJ5_9BACI, A0A2N5H854_9BACI, A0A160ISE8_9BACI, A0A217SRN1_LACSH, A0A1M4TLQ2_9BACL, A0A4R2QSJ5_9BACL, A0A3L7K5H6_9BACI, A0A2N5M452_9BACI, W4QKM5_9BACI, A0A4R2PAA5_9BACL, A0A0J1IMN1_BACCI, R9C857_9BACI, A0A0M4FX23_9BACI, A0A165XSR5_9BACI, A0A179SV99_9BACI, A0A1Y0IS88_9BACL, A0A248TLE9_9BACI, A0A1HOWI33_9BACI, A0A0H4PIL5_9BACI, 18AMT2_9BACI, A0A0D6ZAA3_9BACI, A0A3T0I1Q1_9BACI, A0A1I0SQQ4_9BACI, 13EAA8_BACMM, A0A0M0GB29_SP0GL, A0A1L8ZLZ8_9BACI, A0A370GBM9_9BACI, A0A433H928_9BACI, A0A4R6U795_9BACI, A0A060LXS4_9BACI, A0A074LME5_9BACL, A0A0K9GWU6_9BACI, A0A150KM63_9BACI, K6CV08_BACAZ, A0A323TXM3_9BACI, A0A2N0Z9Q2_9BACI, J8AK67_BACCE, A0A073KUP4_9BACI, A0A292YQZ8_9BACL, A0A226QLR6_9BACI, A0A160FBJ9_9BACI, C3BPR4_9BACI, E6TXR1_BACCJ, A0A1L3MQ53_9BACI, A0A0C2YCQ6_BACBA, Q8EQ49_0CEIH, A0A316D8M3_9BACL, A0A0J6FU61_9BACI, A0A1H8C0C3_9BACI, A0A084J373_BACMY, A0A1I4JPZ3_9BACI, A0A0M2SG37_9BACI, A0A150MMS1_9BACI, A0A1J6WGW3_9BACI, A0A0P6W2Q8_9BACI, A0A110SZE6_9BACI, A7GSJ0_BACCN, A0A2C9Z3P6_BACHU, A0A398BG15_9BACI, A0A0V8JFI7_9BACI, A0A115NPW8_9BACI, A0A4R2B866_9BACI, A0A023DE04_9BACI, A0A023CLR0_9BACI, A0A327YN47_9BACI, A0A0Q9XV74_9BACI, A0A147K7R5_9BACI, A0A443J408_9BACI, A0A498DDK1_9BACI, A0A0K6GMP9_9BACI, A0A429XD58_9BACI, A0A1I1ZLD5_9BACI, A4IQR2_GE0TN, A0A073K3V0_9BACI, A0A1X7D063_9BACI, Q5WF68_BACSK, A0A3S4RLT9_9BACI, A0A150JT68_BACC0, F5L3H6_CALTT, A0A0M0GPU2_9BACI, S5Z7C0_BACPJ, A0A1Z2V3H9_9BACI, A0A3A9KGU4_9BACI, A0A285CLU9_9BACI, A0A366XYH1_9BACI, A0A0D8BRF6_GE0KU, A0A265NFG5_9BACI, A0A428N868_9BACI, A0A2P8HAG1_9BACI, A0A1H0B3U0_9BACI, A0A150M7C5_9BACI, A0A1G8D1C3_9BACI, A0A1G8BRD8_9BACI, A0A4Q4IIH6_9BACL, A0A4Y9AEG4_9BACI, A0A110FQG9_9BACI, A0A0F5HWK7_9BACI, A0A1H1BJ69_9BACI, W9A8X3_9BACI, A0A1H9LW77_9BACI, A0A494Z0K1_9BACI, A0A1M5CXL0_9BACI, A0A1G8JJN9_9BACI, A0A1G6IGH8_9BACI, A0A4Y7S8L6_9FIRM, A0A1X9MFG7_9BACI, A0A0A2UZF1_9BACI, A0A1H9ZLP9_9BACI, A0A1M4XLK4_9CLOT, A0A0A5GIF6_9BACI, A0A0C2VIM1_9BACL, A0A2A2IDA3_9BACI, A0A366EJ45_9BACI, A0A317KZA9_9BACI, A0A0A5GEQ9_9BACI, A0A1E5LK88_9BACI, A0A2S5GEL8_9BACL, A0A1G9LM94_9BACI, A0AlN6PFX1_9BACI, A0A1E7DMX2_9BACI, N4WSS3_9BACI, A0A1I1T1Z9_9BACI, A0A0A1MZ98_9BACI, A0A4R3N0Q4_9BACI, A0A4Y8KST9_9BACL, A0A2U1K6N5_9BACI, A0A075LLD7_9BACI, A0A110V6R2_9BACI, A0A0U1KL95_9BACI, A0A2P6MK99_9BACI, C8WXF8_ALIAD, A0A1M6KIQ3_9CLOT, A0A1L8CTW3_9THEO, A0A1V2A9Q9_9BACI, A0A2T4UAN8_9BACI, A0A4Z0GKV9_9BACL, A0A1M616U1_9FIRM, A0A1I2VPT9_9BACL, A0A1M6S6D3_9BACL, A0AlN7KMH0_9BACL, A0A140L8E0_9CLOT, A0A090J299_9BACI, V6IWU0_9BACL, A0A024P5H3_9BACI, A0A285NM88_9BACI, A0A143MRA0_9BACI, A0A0A5GCA3_9BACI, A0A0U1QSI9_9BACL, A0A0B5AS70_9BACL, A0A1M6C4X1_9CLOT, A0A2I0QX97_9BACI, A0A0P9EJT7_9BACL, A0A1U7MLA0_9FIRM, A0A2T0BRS8_9CLOT, A0A1H2T3C9_9BACL, A0A1H9A7M5_9BACI, A0A084JIX0_9CLOT, D9SLV6_CLOC7, A0A4R2RWP5_9FIRM, U2CLF1_9FIRM and A0A1G8VG90_9BACI. Particularly preferred according to the invention are wild type Spo0A protein sequences and corresponding spoOA genes coding therefor which have at least 55%, more preferably at least 60%, more preferably at least 62%, more preferably at least 70%, even more preferably 80-100% and even more preferably 95-100% sequence identity to the amino acid sequence given by Uniprot identifier A0A074LZY6_PAEPO. When not considering the specific mutations to the Spo0A protein sequence described according to the invention, the mutant Spo0A protein preferably differs from the amino acid sequence given by Uniprot identifier A0A074LZY6_PAEPO by 0-20 amino acids, more preferably 0-15 amino acids, even more preferably 0-10 amino acids, even more preferably 1-5 amino acids, wherein those differences preferably conform to the constraints according to FIG. 5. If the mutant Spo0A sequence, when aligned to the sequence according to Uniprot identifier A0A074LZY6_PAEPO, is longer than said sequence, then each C- or N-terminal extension is preferably no longer than 30 amino acids, more preferably 0-10 amino acids.

The microorganism according to the present invention can comprise, in addition to the mutant degU gene and/or the mutant degS gene, the mutant Spo0A protein or spoOA gene. The microorganism can also comprise, in addition to a mutant DegU protein and/or a mutant DegS protein, a mutant Spo0A protein or spoOA gene.

The microorganism of the present invention exhibits increased and/or stabilised exopolysaccharide production and/or reduced exopolysaccharide degradation relative to an exopolysaccharide producing control strain lacking the mutations of the present invention (“parent”). However, the invention is not limited to the modification of exopolysaccharide producing microorganisms. The invention beneficially also allows to create a microorganism which exhibits increased and/or stabilised exopolysaccharide production and/or reduced exopolysaccharide degradation after those one or more genes for expoloysaccharide production have been introduced that had previously been missing. In particular, the invention allows to prepare a Firmicutes strain for exopolysaccharide production by introducing the degU, degS and/or spoOA of the present invention, such that after further introduction of a heterologous exopolysaccharide gene cluster or modification of native genes the strain is capable of producing one or more desired exopolysaccharides while exhibiting increased and/or stabilised exopolysaccharide production and/or reduced exopolysaccharide degradation.

One of the advantages of the present invention is that the increase or stabilisation of exopolysaccharide production and/or prevention or reduction of exopolysaccharide degradation is rendered possible by mutations in genes readily accessible in a wide variety of Firmicutes microorganisms. Thus, even though the invention is described by way of specific examples below, the methods described in the examples can be transferred to other microorganism species to increase and/or stabilise exopolysaccharide production and/or reduce exopolysaccharide degradation. Furthermore, the mutations described herein do not require the deletion or insertion of large nucleic acid fragments, which could affect transcription of downstream genes. Instead of having to change transcription regulator binding sites of various genes involved in exopolysaccharide formation or deletion of those genes, the present invention allows to achieve the advantages described herein by mutations of the degU, degS and/or spoOA genes. These genes are involved in a complex web of gene regulation pathways, and the effects caused by introducing the mutations of the present invention was not predictable. Likewise, exopolysaccharide production and degradation is subject to complex regulation mechanisms on its own, which is essentially unpredictable. Furthermore, it was particularly surprising that mutations in the degS, degU and/or spoOA genes would lead to an increase or stabilisation of exopolysaccharide production and/or prevention or reduction of exopolysaccharide degradation in the light of publication WO2019221988. This document employs a strain with a Spo0A deficient background and comprising mutated degU and degS genes in addition to the Spo0A deficiency with the explicit ambit to reduce viscosity caused by exopolysaccharides during fermentation. However, as shown herein and contrary to the prima facie plausible teaching of this document, the introduction, in a wild type microorganism, of mutations only in the degU, degS and/or spoOA genes increases or stabilises exopolysaccharide production and/or prevents or reduces exopolysaccharide degradation. Thus, the effects described in said publication are not causally linked to the mutations of the degU, degS and spo0A genes.

The present invention allows, as a particularly notable advantage, to stabilise exopolysaccharides during fermentative production and/or to reduce exopolysaccharide degradation by the microorganism, in particular during fermentative production. This is detectable in a comparative batch fermentation analysis. In such analysis, a batch fermentation of a microorganism according to the present invention and a control fermentation using the appropriate control microorganism are performed and the time of maximum carbon transfer rate is determined. From that time onwards, viscosity of each fermentation broth is measured at predetermined intervals for the next 48h. The sum of viscosity readings of the fermentation of the microorganism of the present invention is then found to be higher than the sum of the same number of viscosity readings for the comparative fermentation. Exopolysaccharides can be used by microorganisms as a reserve carbon or energy source in late fermentation stages as shown in the examples. Thus, even high quantities of exopolysaccharides produced during early fermentation stages can be degraded in later fermentation states or during subsequent storing and downstream processing, thereby reducing the yield of exopolysaccharides and changing product composition, particularly where the product comprises the exopolysaccharide together with further components, preferably one or more of the microorganism, spores thereof and target substances produced by the microorganism, preferably one or more fusaricidins. The invention, however, allows to maintain high viscosity and thus a high exopolysaccharide content throughout fermentation and even possibly in further downstream processing or storage. Thus, compared to wild type strains, the fermenter does not have to be harvested prematurely to balance exopolysaccharide yield against the yield of said further components. Effectively, the invention can be used to produce two product categories by a single fermentation, i.e. the one or more exopolysaccharides and said one or more further components.

Preferably the target fermentation product is or comprises a lipopeptide and/or siderophore and preferably has antimicrobial, preferably antifungal, activity. Preferably the lipopeptides are non-ribosomal lipopeptides (NRPs). Even more preferably the lipopeptides and/or siderophores comprise one or more antimicrobial agents of any of the following types: aculeacin, amphisin, amphomycin, anticapsin, aspartocin, bacillaene, bacillibactin, bacillomycin, bacillorin, bacilysin, bacitracin, caspofungin, cerexin, cichofactin, cormycin, crystallomycin, daptomycin, difficidin, ecomycin, entolysin, fengycin, friulimicin, fusaricidin, gatavalin, hodersin, iturin, jolipeptin, kurstakin, laspartomycin, lichenysin, locillomycin, lokisin, macrolactin, maribasin, marihysin, massetolide, octapeptin, orfamide, paenibacterin, paenilarvin, paeniproxilin, paeniserin, pelgipeptin, plantazolicin, plipastatin, pneumocandin, polyketide, polymyxin, polypeptin, pseudodesmin, pseudomycins, pseudophomin, putisolvin, saltavalin, surfactin, syringafactin, syringomycin, syringopeptin, tensin, tolaasin, tridecaptin, tsushimycin, viscosin and viscosinamide. In particular, the production of antimicrobial target fermentation products obtainable form Paenibacilllus strains is described in WO2016154297. Particularly preferred target fermentation products are fusaricidins. Fusaricidins are a group of antimicrobial substances isolated from Paenibacillus spp. from the class of cyclic lipodepsipeptides which often share the following structural features: a macrocyclic ring consisting of 6 amino acid residues, three of which are L-Thr, D-allo-Thr and D-Ala, as well as the 15-guanidino-3-hydroxypentadecanoic acid tail attached to the N-terminal L-Thr residue by an amide bond (ChemMedChem 7, 871-882, 2012; J. Microbiol. Meth. 85, 175-182, 2011). These compounds are cyclized by a lactone bridge between the N-terminal L-Thr hydroxyl group and the C-terminal D-Ala carbonyl group. The position of the amino acid residues within the depsipeptide cycle are usually numbered starting with the abovementioned L-Thr which itself also carries the GHPD chain and ending with the C-terminal D-Ala. Non-limiting examples of fusaricidins isolated from Paenibacillus are designated LI-F03, LI-F04, LI-F05, LI-F07 and LI-F08 (J. Antibiotics 40 (11), 1506-1514, 1987; Heterocycles 53 (7), 1533-1549, 2000; Peptides 32, 1917-1923, 201 1) and fusaricidins A (also called LI-F04a), B (also called LI-F04b), C (also called LI-F03a) and D (also called LI-F03b) (J. Antibiotics 49 (2), 129-135, 1996; J. Antibiotics 50 (3), 220-228, 1997). The amino acid chain of a fusaricidin is not ribosomally generated but is generated by a non-ribosomal peptide synthetase. Among isolated fusaricidins, fusaricidin A has shown the most promising antimicrobial activity against a variety of clinically relevant fungi and gram-positive bacteria such a Staphylococcus aureus (MIC value range: 0.78-3.12 g/ml) (ChemMedChem 7, 871-882, 2012). Fusaricidins A, B, C and D are also reported to inhibit plant pathogenic fungi such as Fusarium oxysporum, Aspergillus niger, Aspergillus oryzae, and Penicillium thomii (J. Antibiotics 49 (2), 129-135, 1996; J. Antibiotics 50 (3), 220-228, 1997). Fusaricidins such as Li-F05, LI-F07 and LI-F08 have been found to have certain antifungal activity against various plant pathogenic fungi such as Fusarium moniliforme, F. oxysporum, F. roseum, Giberella fujkuroi, Helminthosporium sesamum and Penicillium expansum (J. Antibiotics 40 (11), 1506-1514, 1987). Fusaricidins also have antibacterial activity to Gram-positive bacteria including Staphylococcus aureus (J. Antibiotics 49, 129-135, 1996; J. Antibiotics 50, 220-228, 1997). In addition, fusaricidins have antifungal activity against Leptosphaeria maculans which causes black root rot of canola (Can. J. Microbiol. 48, 159-169, 2002). Moreover, fusaricidins A and B and two related compounds thereof produced by certain Paenibacillus strains were found to induce resistance reactions in cultured parsley cells and to inhibit growth of Fusarium oxysporum (WO2006/016558; EP 1788074A1). In WO2016/020371 it was found that the whole culture broth, the culture medium and cell-free extracts of the bacterial strains Lu16774, Lu17007 and Lu17015 show inhibitory activity inter alia against Alternaria spp., Botrytis cinerea and Phytophthora infestans.

The microorganism according to the present invention preferably comprises a mutant degU gene, wherein the degU gene codes for a DegU protein having reduced DNA binding activity and/or lacks a functional DNA binding domain. This is preferably achieved by providing a mutant degU gene coding for a mutant DegU protein, wherein the mutation affects the a LuxR-type DNA-binding HTH domain (PF00196). As shown below in the examples, the mere provisioning of a mutant degU gene already is sufficient to improve exopolysaccharide production and/or reduce exopolysaccharide degradation during a fermentation process. This was particularly surprising in view of WO2019221988, which explicitly declares that such mutations of the degU gene do not lead to an increase in viscosity of the fermentation broth and thus are not suitable for increasing and/or stabilising exopolysaccharide production and/or reducing exopolysaccharide degradation during fermentation.

It is a further advantage that the DegS, DegU and DegS+DegU mutants of the present invention do not abolish or significantly reduce the microorganism's capability of sporulation. This is a particular advantage for spore forming plant health compositions or other applications which rely on spore formation.

The DegU protein preferably has a reduced DNA binding activity and/or lacks a functional DNA binding domain. The presence of these traits can be easily identified in the microorganism of the present invention, preferably of genus Paenibacillus, by observing an increase in viscosity or a retaining of viscosity during fermentation compared to the corresponding wild type strain.

As described above, the wild type DegU protein comprises a DNA-binding HTH domain; this domain extends, according to the numbering of the protein sequence with Uniprot identifier E3EBP5, from amino acid position 171 to the end of the sequence. Further information on the DNA binding domain is available from the corresponding Pfam and InterPro databases. For example, for the most preferred wild type DegU protein sequence E3EBP5 the DNA-binding domain is predicted to comprise 4 alpha-helix domains, spanning the positions 180-191, 195-202, 206-221 and 225-235. It is preferred if the DegU protein DNA-binding domain is mutated in the third or fourth, most preferably in the third alpha helical domain. Here, mutations in the protein sequence will generally not influence correct folding and functioning of the remainder of the DegU protein.

Preferably, the DegU protein mutation comprises or consists of, in decreasing order of preference for each alternative a) and b), one or more of:
a) Q218*, Q218K, Q218N, Q218D, Q218R,
b) D223*, D223*+M220N, D223*+M220N+E221G, D223*+M220N+V222G, D223*+M220N+E221G+V222G, D223*+M220D, D223*+M220E, D223*+M220H, D223*+M220F, D223*+M220W, D223*+M220S, D223*+M220A.

For the purposes of the present invention, the aforementioned numbering is with reference to the wild type DegU protein sequence of Uniprot identifier E3EBP5. It is to be noted, as indicated above, that the mutant DegU protein, when disregarding the above mentioned specifically listed mutations, has at least 45%, more preferably at least 51%, more preferably at least 54% and even more preferably 73-100% sequence identity to the amino acid sequence given by Uniprot identifier E3EBP5_PAEPS.

Both mutations a) and b) simultaneously fall within the third predicted alpha helix of the DNA binding domain. As shown in the examples, mutations of both type a) and b) both result in an increase of fermentation broth viscosity over the course of a fermentation. Furthermore, even though viscosity of the fermentation broth varies in late fermentation stages, it does not fall below 50% of the maximal viscosity of the fermentation broth of the wild type strain, as shown in the examples, and preferably doesn't fall below 70% of the maximal viscosity of the fermentation broth of the wild type strain, measured at 48h after the point in time with the maximal carbon transfer rate during fermentation. Conditions for measuring viscosity are given in the examples below.

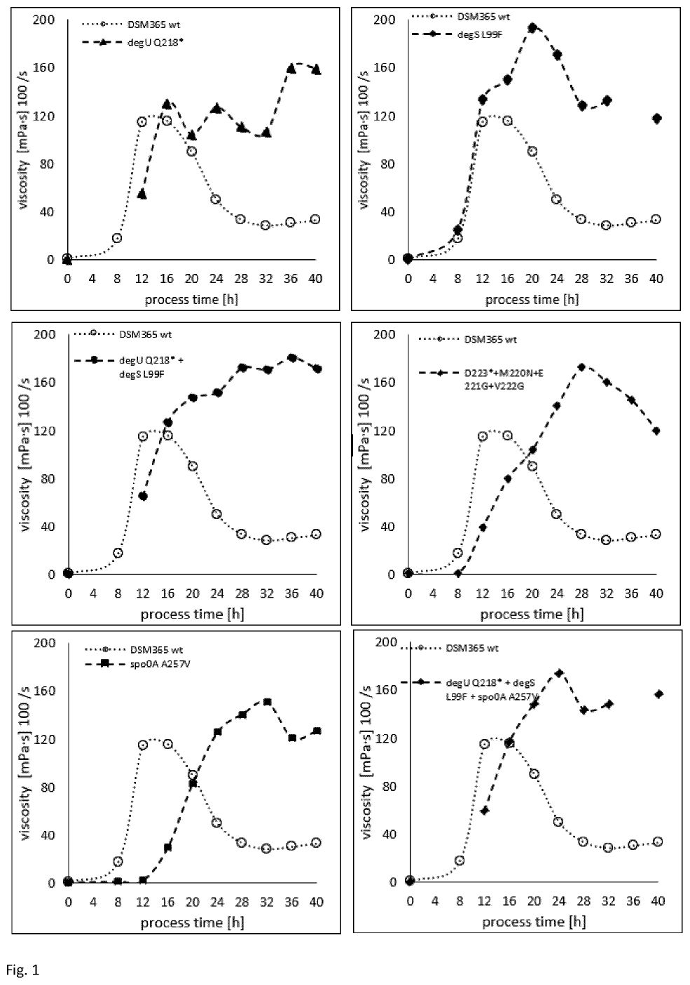

Preferably, mutations of alternative a) result in, compared to microorganisms comprising only a mutation according to alternative b), a faster increase in viscosity such that the plateau of viscosity is reached at an earlier time compared to microorganisms comprising only a mutation according to alternative b). On the other hand, microorganisms comprising a DegU mutation according to alternative b) preferably provide a higher peak viscosity compared to those according to alternative a), see also FIG. 1.

The mutated amino acids according to alternative a) and b), respectively, are listed above in increasing order of their respective frequency in natural homologs of DegU proteins. As the invention is interested in providing microorganisms with altered properties of the DegU protein compared to the wild type, the most infrequent alteration is the most preferred one, and preference decreases with increasing frequency of the respective amino acid at the respective position.

With the exception of mutation Q218* the aforementioned DegU protein mutations can also be combined. Thus, the invention also pertains to microorganisms comprising a mutant degU gene coding for a mutant DegU protein, wherein the mutation comprises or consists of any of Q218K+D223* Q218K+M220N+D223*, Q218K+M220N+E221G+D223*, Q218K+M220N+V222G+D223*, Q218K+M220N+E221G+V222G+D223*, Q218K+M220D+D223*, Q218K+M220E+D223*, Q218K+M220H+D223*, Q218K+M220F+D223*, Q218K+M220W+D223*, Q218K+M220S+D223*, Q218K+M220A+D223*, Q218N+D223*, Q218N+M220N+D223*, Q218N+M220N+E221G+D223*, Q218N+M220N+V222G+D223*, Q218N+M220N+E221G+V222G+D223*, Q218N+M220D+D223*, Q218N+M220E+D223*, Q218N+M220H+D223*, Q218N+M220F+D223*, Q218N+M220W+D223*, Q218N+M220S+D223*, Q218N+M220A+D223*, Q218D+D223*, Q218D+M220N+D223*, Q218D+M220N+E221G+D223*, Q218D+M220N+V222G+D223*, Q218D+M220N+E221G+V222G+D223*, Q218D+M220D+D223*, Q218D+M220E+D223*, Q218D+M220H+D223*, Q218D+M220F+D223*, Q218D+M220W+D223*, Q218D+M220S+D223*, Q218D+M220A+D223*, Q218R+D223*, Q218R+M220N+D223*, Q218R+M220N+E221G+D223*, Q218R+M220N+V222G+D223*, Q218R+M220N+E221G+V222G+D223*, Q218R+M220D+D223*, Q218R+M220E+D223*, Q218R+M220H+D223*, Q218R+M220F+D223*, Q218R+M220W+D223*, Q218R+M220S+D223*, Q218R+M220A+D223*, in the numbering of the sequence according to the Uniprot identifier E3EBP5.

The microorganism according to the present invention preferably comprises a mutant degS gene, wherein the degS gene codes for a DegS protein lacking a functional single binding domain, a functional phosphoacceptor domain and/or a functional ATPase domain. As shown below in the examples, the mere provisioning of a mutant degS gene already is sufficient to improve exopolysaccharide production and/or reduce exopolysaccharide degradation during a fermentation process. This was particularly surprising in view of WO2019221988, which explicitly declares that such mutations of the degS gene do not lead to an increase in viscosity of the fermentation broth and thus are not suitable for increasing and/or stabilising exopolysaccharide production and/or reducing exopolysaccharide degradation during fermentation.

The DegS protein preferably lacks a functional single binding domain, a functional phosphoacceptor domain and/or a functional ATPase domain. The presence of these traits can be easily identified in the microorganism of the present invention, preferably of genus Paenibacillus, by observing an increase in viscosity or a retaining of viscosity during fermentation compared to the corresponding wild type strain, and can be easily achieved, for example by introducing a mutation in the sensor DegS domain (IPR008595).

As described above, the wild type DegS protein comprises a Sensor DegS domain; this domain extends, according to the numbering of the protein sequence with Uniprot identifier A0A074LBY4_PAEPO, from amino acid position 10 to 165. Further information on the DNA binding domain is available from the corresponding Pfam and InterPro databases. For example, for the most preferred wild type DegS protein sequence A0A074LBY4_PAEPO the DNA-binding domain is predicted to comprise 2 alpha-helix domains, spanning the positions 5-81 and 84-186, wherein the amino acids of positions 175-186 already overlap with the histidine kinase domain. It is preferred if the DegS protein DNA-binding domain is mutated such that the overall alpha-helical structure remains intact to prevent interference with the folding of the histidine kinase domain.

Preferably, the mutant DegS protein differs from the corresponding wild type sequence by one or more mutations selected from, in decreasing order of preference, L99F, L99C, L99D, L99E, L99G, L99H, L99K, L99N, L99P, L99Q, L99R, L99S, L99W or L99Y.

For the purposes of the present invention, the aforementioned numbering is with reference to the wild type DegS protein sequence of Uniprot identifier A0A074LBY4_PAEPO. It is to be noted, as indicated above, that the mutant DegS protein, when disregarding the above mentioned specifically listed mutations, has at least 40%, more preferably at least 46%, more preferably at least 58% and even more preferably 80-100% sequence identity to the amino acid sequence given by Uniprot identifier A0A074LBY4_PAEPO.

The aforementioned specific mutations fall within the second predicted alpha helix of the DegS Sensor domain. As shown in the examples, such mutations both result in an increase of fermentation broth viscosity over the course of a fermentation. Furthermore, even though viscosity of the fermentation broth varies in late fermentation stages, it does not fall below 50% of the maximal viscosity of the fermentation broth of the wild type strain, as shown in the examples, and preferably doesn't fall below 70% of the maximal viscosity of the fermentation broth of the wild type strain. Conditions for measuring viscosity are given in the examples below.

Preferably, mutations of the degS gene according to the invention result in a higher peak viscosity compared to the wild type strain; further preferably the increase in viscosity is not delayed compared to the wild type strain such that at the time of maximum viscosity of a wild type fermentation broth, the viscosity of the corresponding DegS mutant fermentation broth is at least 90% of that of the wild type fermentation broth, and preferably is 100%-300% of the wild type fermentation broth (see also FIG. 1).

The mutated amino acids for the DegS mutant protein are listed above in increasing order of their respective frequency in natural homologs of DegS proteins. As the invention is interested in providing microorganisms with altered properties of the DegS protein compared to the wild type, the most infrequent alteration is the most preferred one, and preference decreases with increasing frequency of the respective amino acid at the respective position.

The microorganism according to the present invention preferably comprises a mutant spoOA gene, wherein the mutation is located in the DNA binding or receiver domain and results in a reduction or elimination of phosphorylation of the Spo0A protein. As shown below in the examples, the mere provisioning of a mutant spo0A gene is already sufficient to improve exopolysaccharide production and/or reduce exopolysaccharide degradation during a fermentation process. This was particularly surprising in view of WO2019221988 and WO2016154297. According to the latter publication, the parent strain of all DegU/DegS mutants of WO2019221988 did already comprise a spo0A mutation. In WO2016154297, this spoOA mutation was described as a reason for a stable colony morphotype, whereas its parent strain showed varying morphologies such as a viscous, mucoid phenotype. In contrast to our observations using a wildtype strain for targeted insertion of point mutations, in WO2019221988 no increase or stabilization of the viscosity from fermentation brothes was observed, especially in the light that an already randomly mutated strain was used as the parent strain for further genetic optimization in the latter document.

The mutant Spo0A protein preferably lacks a functional DNA binding or receiver domain. The presence of these traits can be easily identified in the microorganism of the present invention, preferably of genus Paenibacillus, by observing an increase in viscosity or a retaining of viscosity during fermentation compared to the corresponding wild type strain, and can be easily achieved, for example by introducing a mutation in the Spo0A C-terminal domain (IPR014879).

As described above, the wild type Spo0A protein comprises a Sporulation initiation factor Spo0A C terminal domain; this domain extends, according to the numbering of the protein sequence with Uniprot identifier A0A074LZY6_PAEPO, from amino acid position 158 to 261. Further information on the Spo0A C-terminal domain is available from the aforementioned corresponding Pfam and InterPro databases.

Preferably, the mutation of the mutant Spo0A protein consists of or comprises any of
A257V, more preferably A257S, or
I161R, more preferably I161L, or
in decreasing order of preference: A257S+I161I, A257A+I161L, A257V+I161I, A257S+I161F or A257A+I161R.

For the purposes of the present invention, the aforementioned numbering is with reference to the wild type Spo0A protein sequence of Uniprot identifier A0A074LZY6_PAEPO. It is to be noted, as indicated above, that the mutant Spo0A protein, when disregarding the above mentioned specifically listed mutations, has at least 55%, more preferably at least 60%, more preferably at least 62%, more preferably at least 70%, even more preferably 80-100% and even more preferably 95-100% sequence identity to the amino acid sequence given by Uniprot identifier A0A074LZY6_PAEPO.

Preferably, the mutant Spo0A protein comprises one of the two aforementioned mutations at position 257, i.e. A257V or, more preferably, A257S. This position falls within the last predicted alpha helix of the Spo0A C-terminal domain. Also, preferably the mutant Spo0A protein comprises one of the two aforementioned mutations at position 161, i.e. I161R or, more preferably I161L. This position falls within the first predicted alpha helix of the Spo0A C-terminal domain. Further preferably the mutant Spo0A protein comprises any of the aforementioned respective mutations at each of the aforementioned positions, i.e., in decreasing order of preference: A257S+I161I, A257A+I161L, A257V+I161I, A257S+I161F or A257A+I161R. The mutated amino acids of the double mutants are listed in increasing order of their respective frequency in natural homologs of Spo0A proteins. As the invention is interested in providing microorganisms with altered properties of the Spo0A protein compared to the wild type, the most infrequent alteration is the most preferred one, and preference decreases with increasing frequency of the respective amino acid at the respective position.

Typically the aforementioned mutations of the Spo0A protein result in, compared to the wild type, a delay before maximum fermentation broth viscosity is achieved. Furthermore, even though viscosity of the fermentation broth varies in late fermentation stages, it does not fall below 50% of the maximum viscosity of the fermentation broth of the wild type strain, as shown in the examples, and preferably doesn't fall below 70% of the maximal viscosity of the fermentation broth of the wild-type strain. Conditions for measuring viscosity are given in the examples below.

As shown in the examples, providing microorganisms comprising mutants in two genes instead of merely one gene can further improve the advantages obtainable according to the invention and in particularly lead to an increased and/or stabilised exopolysaccharide production and/or reduced exopolysaccharide degradation.

Furthermore, the invention provides microorganisms comprising both a mutant degU gene and mutant degS gene, a mutant degU gene and a mutant spoOA gene, a mutant degS gene and a mutant spo0A gene or a mutant degU gene, a mutant degS gene and a mutant spoOA gene. As shown exemplarily in the examples, providing microorganisms comprising mutants in two genes instead of merely one gene can further improve the advantages obtainable according to the invention and in particularly lead to an increased and/or stabilised exopolysaccharide production and/or reduced exopolysaccharide degradation. For example, a microorganism comprising both a mutant DegU protein and mutant DegS protein allows to achieve a more steady viscosity in late fermentation stages. As also shown in the examples, a microorganism comprising a mutant DegU protein, a mutant DegS protein and a mutant Spo0A protein also allows to achieve an earlier maximum viscosity compared to the corresponding Spo0A single gene mutant microorganism. Also, as an example, a microorganism with a mutant DegS can reach significantly higher viscosity compared to a strain with wt-degS gene.

According to the invention it is particularly preferred that the microorganism, when grown in a liquid fermentation medium, causes a viscosity increase of the fermentation medium such that the fermentation medium viscosity remains higher than 50%, preferably at least 60%, more preferably at least 70%, more preferably at least 80% of the maximum fermentation medium viscosity obtained in a fermentation of the corresponding wild type strain within 48h after maximal carbon transfer rate in a batch fermentation. Thus, the exopolysaccharide production of the microorganism of the present invention is advantageously increased and/or the exopolysaccharide degradation is advantageously reduced.

The microorganism according to the present invention preferably is selected from the taxonomic rank of
phylum Firmicutes, class Bacilli, Clostridia or Negativicutes,
more preferably of order Bacillales, Clostridiales, Thermoanaerobacterales, Thermosediminibacterales or Selenomonadales,
more preferably of family Bacillaceae, Paenibacillaceae, Pasteuriaceae, Clostridiaceae, Peptococcaceae, Heliobacteriaceae, Syntrophomonadaceae, Thermoanaerobacteraceae, Tepidanaerobacteraceae or Sporomusaceae, more preferably of genus Alkalibacillus, Bacillus, Geobacillus, Halobacillus, Lysinibacillus, Piscibacillus, Terribacillus, Brevibacillus, Paenibacillus, Thermobacillus, Pasteuria, Clostridium, Desulfotomaculum, Heliobacterium, Pelospora, Pelotomaculum, Caldanaerobacter, Moorella, Thermoanaerobacter, Tepidanaerobacter, Propionispora or Sporomusa, more preferably of genus Bacillus, Paenibacillus or Clostridium.

Particularly microorganisms of the families Bacillaceae, Paenibacillaceae and Clostridiaceae are known to produce exopolysaccharides and are important microorganisms in industrial fermentation processes. Furthermore, among microorganisms of such genera are known spore producers.

In agriculture, bacterial spores were used in plant pest control compositions reducing or preventing phytopathogenic fungal or bacterial diseases. Spore biologicals are also applied to improve plants resistance against biotic and abiotic stress, to accelerate the growth of the plant and to increase the yield during plant, fruit or legume harvest. Spore products were applied to leaves, shoots, fruits, roots or plant propagation material as well as to the substrate where the plants are to grow (Toyota K. Bacillus-related Spore Formers: Attractive Agents for Plant Growth Promotion. Microbes Environ. 2015; 30 (3): 205-207. doi: 10.1264/jsme2.me3003rh). Bochow, H., et al. “Use of Bacillus Subtilis as Biocontrol Agent. IV. Salt-Stress Tolerance Induction by Bacillus Subtilis FZB24 Seed Treatment in Tropical Vegetable Field Crops, and Its Mode of Action/Die Verwendung von Bacillus Subtilis zur biologischen Bekämpfung. IV. Induktion einer Salzstress-Toleranz durch Applikation von Bacillus subtilis FZB24 bei tropischem Feldgemüse und sein Wirkungsmechanismus.” Zeitschrift für Pflanzenkrankheiten und Pflanzenschutz/Journal of Plant Diseases and Protection, vol. 108, no. 1, 2001, pp. 21-30. JSTOR, www.jstor.org/stable/43215378. Accessed 14 Dec. 2020.) (Hashem, Abeer & Tabassum, B. & Abd_Allah, Elsayed. (2019). Bacillus subtilis: A plant-growth promoting rhizobacterium that also impacts biotic stress. Saudi Journal of Biological Sciences. 26. 10.1016/j.sjbs.2019.05.004.)

Furthermore, bacterial spores were applied in the area of nanobiotechnology and building chemistry such as for self-healing concrete (crack healing), mortar stability and reduced water permeability [J. Y. Wang, H. Soens, W. Verstraete, N. De Belie, Self-healing concrete by use of microencapsulated bacterial spores, Cement and Concrete Research, Volume 56, 2014, 139-152, ISSN 0008-8846, https://doi.org/10.1016/j.cemconres.2013.11.009] [Ricca E, Cutting S M. Emerging Applications of Bacterial Spores in Nanobiotechnology. J Nanobiotechnology. 2003; 1 (1): 6. Published 2003 Dec. 15. doi: 10.1186/1477-3155-1-6].

Additionally, bacterial spores were applied in the area of cleaning products, such as for cleaning of laundry, hard surfaces, sanitation and odor control (Caselli E. Hygiene: microbial strategies to reduce pathogens and drug resistance in clinical settings. Microb Biotechnol. 2017 September; 10 (5): 1079-1083. doi: 10.1111/1751-7915.12755. Epub 2017 Jul. 5) in the clinical and domestic setting. As an example, spores were used in cosmetic compositions such as skin cleaning products (US20070048244), for dishwashing agents (WO2014/107111), pipe degreasers (DE19850012), malodor control of laundry (WO2017/157778 and EP3430113) or the removal of allergens (US20020182184). Spores can also be embedment into non-biogenic matrices to catalyze subsequent matrix breakdown.

In addition, bacterial spores were applied in the area of human and animal nutrition and health. As an example different bacterial strains were applied to broilers as part of antibiotic replacement strategy (Neveling, D. P., Dicks, L. M. Probiotics: an Antibiotic Replacement Strategy for Healthy Broilers and Productive Rearing. Probiotics & Antimicro. Prot. 13, 1-11 (2021). https://doi.org/10.1007/s12602-020-09640-z). Other examples include aquaculture, pigs and many more (Nayak, S. K. (2021), Multifaceted applications of probiotic Bacillus species in aquaculture with special reference to Bacillus subtilis. Rev. Aquacult., 13:862-906. https://doi.org/10.1111/raq.12503). Applications of bacterial spored for human health are also widely described (e.g. US20180289752; Lee, N K., Kim, W S. & Paik, H D. Bacillus strains as human probiotics: characterization, safety, microbiome, and probiotic carrier. Food Sci Biotechnol 28, 1297-1305 (2019). https://doi.org/10.1007/s10068-019-00691-9).

It is thus a particular advantage of the present invention that the mutations described herein allow for an increased and/or stabilised exopolysaccharide production and/or reduced exopolysaccharide degradation in such industrially relevant organisms. With the help of microbial exopolysaccharides, bacterial spores were further protected against biotic and abiotic stress and supported for germination and propagation.

Particularly preferred are microorganisms of one of the following species:

Paenibacillus species: P. abekawaensis, P. abyssi, P. aceris, P. aceti, P. aestuarii, P. agarexedens, P. agaridevorans, P. alba, P. albidus, P. albus, P. alginolyticus, P. algorifonticola, P. alkaliterrae, P. alvei, P. amylolyticus, P. anaericanus, P. antarcticus, P. antibioticophila, P. antri, P. apiaries, P. apiarius, P. apis, P. aquistagni, P. arachidis, P. arcticus, P. assamensis, P. aurantiacus, P. azoreducens, P. azotifigens, P. baekrokdamisoli, P. barcinonensis, P. barengoltzii, P. beijingensis, P. borealis, P. bouchesdurhonensis, P. bovis, P. brasilensis, P. brassicae, P. bryophyllum, P. caespitis, P. camelliae, P. camerounensis, P. campinasensis, P. castaneae, P. catalpae, P. cathormii, P. cavernae, P. cellulosilyticus, P. cellulositrophicus, P. chartarius, P. chibensis, P. chinensis, P. chinjuensis, P. chitinolyticus, P. chondroitinus, P. chungangensis, P. cineris, P. cisolokensis, P. contaminans, P. cookii, P. crassostreae, P. cucumis, P. curdlanolyticus, P. daejeonensis, P. dakarensis, P. darangshiensis, P. darwinianus, P. dauci, P. dendritiformis, P. dongdonensis, P. donghaensis, P. doosanensis, P. durus, P. edaphicus, P. ehimensis, P. elgii, P. elymi, P. endophyticus, P. enshidis, P. esterisolvens, P. etheri, P. eucommiae, P. faecis, P. favisporus, P. ferrarius, P. filicis, P. flagellatus, P. fonticola, P. forsythiae, P. frigoriresistens, P. fujiensis, P. fukuinensis, P. gansuensis, P. gelatinilyticus, P. ginsengagri, P. ginsengarvi, P. ginsengihumi, P. ginsengiterrae, P. glacialis, P. glebae, P. glucanolyticus, P. glycanilyticus, P. gorillae, P. graminis, P. granivorans, P. guangzhouensis, P. harenae, P. helianthi, P. hemerocallicola, P. herberti, P. hispanicus, P. hodogayensis, P. hordei, P. horti, P. humicus, P. hunanensis, P. ihbetae, P. ihuae, P. ihumii, P. illinoisensis, P. insulae, P. intestini, P. jamilae, P. jilunlii, P. kobensis, P. koleovorans, P. konkukensis, P. konsidensis, P. koreensis, P. kribbensis, P. kyungheensis, P. lactis, P. lacus, P. larvae, P. lautus, P. lemnae, P. lentimorbus, P. lentus, P. liaoningensis, P. limicola, P. lupini, P. luteus, P. lutimineralis, P. macerans, P. macquariensis, P. marchantiophytorum, P. marinisediminis, P. marinum, P. massiliensis, P. maysiensis, P. medicaginis, P. mendelii, P. mesophilus, P. methanolicus, P. mobilis, P. montanisoli, P. montaniterrae, P. motobuensis, P. mucilaginosus, P. nanensis, P. naphthalenovorans, P. nasutitermitis, P. nebraskensis, P. nematophilus, P. nicotianae, P. nuruki, P. oceanisediminis, P. odorifer, P. oenotherae, P. oralis, P. oryzae, P. oryzisoli, P. ottowii, P. ourofinensis, P. pabuli, P. paeoniae, P. panacihumi, P. panacisoli, P. panaciterrae, P. paridis, P. pasadenensis, P. pectinilyticus, P. peoriae, P. periandrae, P. phocaensis, P. phoenicis, P. phyllosphaerae, P. physcomitrellae, P. pini, P. pinihumi, P. pinisoli, P. pinistramenti, P. pocheonensis, P. polymyxa, P. polysaccharolyticus, P. popilliae, P. populi, P. profundus, P. prosopidis, P. protaetiae, P. provencensis, P. psychroresistens, P. pueri, P. puernese, P. puldeungensis, P. purispatii, P. qingshengii, P. qinlingensis, P. quercus, P. radicis, P. relictisesami, P. residui, P. rhizoplanae, P. rhizoryzae, P. rhizosphaerae, P. rigui, P. ripae, P. rubinfantis, P. ruminocola, P. sabinae, P. sacheonensis, P. salinicaeni, P. sanguinis, P. sediminis, P. segetis, P. selenii, P. selenitireducens, P. senegalensis, P. senegalimassiliensis, P. seodonensis, P. septentrionalis, P. sepulcri, P. shenyangensis, P. shirakamiensis, P. shunpengii, P. siamensis, P. silagei, P. silvae, P. sinopodophylli, P. solanacearum, P. solani, P. soli, P. sonchi group, P. sophorae, P. spiritus, P. sputi, P. stellifer, P. susongensis, P. swuensis, P. taichungensis, P. taihuensis, P. taiwanensis, P. taohuashanense, P. tarimensis, P. telluris, P. tepidiphilus, P. terrae, P. terreus, P. terrigena, P. tezpurensis, P. thailandensis, P. thermoaerophilus, P. thermophilus, P. thiaminolyticus, P. tianmuensis, P. tibetensis, P. timonensis, P. translucens, P. tritici, P. triticisoli, P. tuaregi, P. tumbae, P. tundrae, P. turicensis, P. tylopili, P. typhae, P. tyrfis, P. uliginis, P. urinalis, P. validus, P. velaei, P. vini, P. vortex, P. vorticalis, P. vulneris, P. wenxiniae, P. whitsoniae, P. wooponensis, P. woosongensis, P. wulumuqiensis, P. wynnii, P. xanthanilyticus, P. xanthinilyticus, P. xerothermodurans, P. xinjiangensis, P. xylanexedens, P. xylaniclasticus, P. xylanilyticus, P. xylanisolvens, P. yanchengensis, P. yonginensis, P. yunnanensis, P. zanthoxyli, P. zeae, preferably P. agarexedens, P. agaridevorans, P. alginolyticus, P. alkaliterrae, P. alvei, P. amylolyticus, P. anaericanus, P. antarcticus, P. assamensis, P. azoreducens, P. barcinonensis, P. borealis, P. brassicae, P. campinasensis, P. chinjuensis, P. chitinolyticus, P. chondroitinus, P. cineris, P. curdlanolyticus, P. daejeonensis, P. dendritiformis, P. ehimensis, P. elgii, P. favisporus, P. glucanolyticus, P. glycanilyticus, P. graminis, P. granivorans, P. hodogayensis, P. illinoisensis, P. jamilae, P. kobensis, P. koleovorans, P. koreensis, P. kribbensis, P. lactis, P. larvae, P. lautus, P. lentimorbus, P. macerans, P. macquariensis, P. massiliensis, P. mendelii, P. motobuensis, P. naphthalenovorans, P. nematophilus, P. odorifer, P. pabuli, P. peoriae, P. phoenicis, P. phyllosphaerae, P. polymyxa, P. popilliae, P. rhizosphaerae, P. sanguinis, P. stellifer, P. taichungensis, P. terrae, P. thiaminolyticus, P. timonensis, P. tylopili, P. turicensis, P. validus, P. vortex, P. vulneris, P. wynnii, P. xylanilyticus, particularly preferred Paenibacillus koreensis, Paenibacillus rhizosphaerae, Paenibacillus polymyxa, Paenibacillus amylolyticus, Paenibacillus terrae, Paenibacillus polymyxa polymyxa, Paenibacillus polymyxa plantarum, Paenibacillus nov. spec epiphyticus, Paenibacillus terrae, Paenibacillus macerans, Paenibacillus alvei, more preferred Paenibacillus polymyxa, Paenibacillus polymyxa polymyxa, Paenibacillus polymyxa plantarum, Paenibacillus nov. spec epiphyticus, Paenibacillus terrae, Paenibacillus macerans, Paenibacillus alvei, even more preferred Paenibacillus polymyxa, Paenibacillus polymyxa polymyxa, Paenibacillus polymyxa plantarum and Paenibacillus terrae. Bacillus species: B. abyssalis, B. acanthi, B. acidiceler, B. acidicola, B. acidiproducens, B. aciditolerans, B. acidopullulyticus, B. acidovorans, B. aeolius, B. aequororis, B. aeris, B. aerius, B. aerolacticus, B. aestuarii, B. aidingensis, B. akibai, B. alcaliinulinus, B. alcalophilus, B. algicola, B. alkalicola, B. alkalilacus, B. alkalinitrilicus, B. alkalisediminis, B. alkalitelluris, B. alkalitolerans, B. alkalogaya, B. altitudinis, B. alveayuensis, B. amiliensis, B. andreesenii, B. andreraoultii, B. aporrhoeus, B. aquimaris, B. arbutinivorans, B. aryabhattai, B. asahii, B. aurantiacus, B. australimaris, B. azotoformans, B. bacterium, B. badius, B. baekryungensis, B. bataviensis, B. benzoevorans, B. beringensis, B. berkeleyi, B. beveridgei, B. bingmayongensis, B. bogoriensis, B. borbori, B. boroniphilus, B. butanolivorans, B. cabrialesii, B. caccae, B. camelliae, B. campisalis, B. canaveralius, B. capparidis, B. carboniphilus, B. casamancensis, B. caseinilyticus, B. catenulatus, B. cavernae, B. cecembensis, B. cellulosilyticus, B. chagannorensis, B. chandigarhensis, B. cheonanensis, B. chungangensis, B. ciccensis, B. cihuensis, B. circulans, B. clausii, B. coagulans, B. coahuilensis, B. cohnii, B. composti, B. coniferum, B. coreaensis, B. crassostreae, B. crescens, B. cucumis, B. dakarensis, B. daliensis, B. danangensis, B. daqingensis, B. decisifrondis, B. decolorationis, B. depressus, B. deramificans, B. deserti, B. dielmoensis, B. djibelorensis, B. drentensis, B. ectoiniformans, B. eiseniae, B. enclensis, B. endolithicus, B. endophyticus, B. endoradicis, B. endozanthoxylicus, B. farraginis, B. fastidiosus, B. fengqiuensis, B. fermenti, B. ferrariarum, B. filamentosus, B. firmis, B. firmus, B. flavocaldarius, B. flexus, B. foraminis, B. fordii, B. formosensis, B. fortis, B. freudenreichii, B. fucosivorans, B. fumarioli, B. funiculus, B. galactosidilyticus, B. galliciensis, B. gibsonii, B. ginsenggisoli, B. ginsengihumi, B. ginsengisoli, B. glennii, B. glycinifermentans, B. gobiensis, B. gossypii, B. gottheilii, B. graminis, B. granadensis, B. hackensackii, B. haikouensis, B. halmapalus, B. halodurans, B. halosaccharovorans, B. haynesii, B. hemicellulosilyticus, B. hemicentroti, B. herbersteinensis, B. hisashii, B. horikoshii, B. horneckiae, B. horti, B. huizhouensis, B. humi, B. hunanensis, B. hwajinpoensis, B. idriensis, B. indicus, B. infantis, B. infernus, B. intermedius, B. intestinalis, B. iocasae, B. isabeliae, B. israeli, B. jeddahensis, B. jeotgali, B. kexueae, B. kiskunsagensis, B. kochii, B. kokeshiiformis, B. koreensis, B. korlensis, B. kribbensis, B. krulwichiae, B. kwashiorkori, B. kyonggiensis, B. lacisalsi, B. lacus, B. lehensis, B. lentus, B. ligniniphilus, B. lindianensis, B. litoralis, B. loiseleuriae, B. lonarensis, B. longiquaesitum, B. longisporus, B. luciferensis, B. luteolus, B. luteus, B. lycopersici, B. magaterium, B. malikii, B. mangrovensis, B. mangrovi, B. mannanilyticus, B. manusensis, B. marasmi, B. marcorestinctum, B. marinisedimentorum, B. marisflavi, B. maritimus, B. marmarensis, B. massiliglaciei, B. massilioanorexius, B. massiliogabonensis, B. massiliogorillae, B. massilionigeriensis, B. massiliosenegalensis, B. mediterraneensis, B. megaterium, B. mesonae, B. mesophilum, B. mesophilus, B. methanolicus, B. miscanthi, B. muralis, B. murimartini, B. nakamurai, B. nanhaiisediminis, B. natronophilus, B. ndiopicus, B. nealsonii, B. nematocida, B. niabensis, B. niacini, B. niameyensis, B. nitritophilus, B. notoginsengisoli, B. novalis, B. obstructivus, B. oceani, B. oceanisediminis, B. ohbensis, B. okhensis, B. okuhidensis, B. oleivorans, B. oleronius, B. olivae, B. onubensis, B. oryzae, B. oryzaecorticis, B. oryzisoli, B. oryziterrae, B. oshimensis, B. pakistanensis, B. panacisoli, B. panaciterrae, B. paraflexus, B. patagoniensis, B. persicus, B. pervagus, B. phocaeensis, B. pichinotyi, B. piscicola, B. piscis, B. plakortidis, B. pocheonensis, B. polygoni, B. polymachus, B. populi, B. praedii, B. pseudalcaliphilus, B. pseudofirmus, B. pseudoflexus, B. pseudomegaterium, B. psychrosaccharolyticus, B. pumilus, B. purgationiresistens, B. qingshengii, B. racemilacticus, B. rhizosphaerae, B. rigiliprofundi, B. rubiinfantis, B. ruris, B. safensis, B. saganii, B. salacetis, B. salarius, B. salidurans, B. salis, B. salitolerans, B. salmalaya, B. salsus, B. sediminis, B. selenatarsenatis, B. senegalensis, B. seohaeanensis, B. shacheensis, B. shackletonii, B. shandongensis, B. shivajii, B. similis, B. simplex, B. sinesaloumensis, B. siralis, B. smithii, B. solani, B. soli, B. solimangrovi, B. solisilvae, B. songklensis, B. spongiae, B. sporothermodurans, B. stamsii, B. subterraneus, B. swezeyi, B. taeanensis, B. taiwanensis, B. tamaricis, B. taxi, B. terrae, B. testis, B. thaonhiensis, B. thermoalkalophilus, B. thermoamyloliquefaciens, B. thermoamylovorans, B. thermocopriae, B. thermolactis, B. thermophilus, B. thermoproteolyticus, B. thermoterrestris, B. thermozeamaize, B. thioparans, B. tianmuensis, B. tianshenii, B. timonensis, B. tipchiralis, B. trypoxylicola, B. tuaregi, B. urumqiensis, B. vietnamensis, B. vini, B. vireti, B. viscosus, B. vitellinus, B. wakoensis, B. weihaiensis, B. wudalianchiensis, B. wuyishanensis, B. xiamenensis, B. xiaoxiensis, B. zanthoxyli, B. zeae, B. zhangzhouensis, B. zhanjiangensis, preferably Bacillus licheniformis, B. megaterium, B. subtilis, B. pumilus, B. firmus, B. thuringiensis, B. velezensis, B. linens, B. atrophaeus, B. amyloliquefaciens, B. aryabhattai, B. cereus, B. aquatilis, B. circulans, B. clausii, B. sphaericus, B. thiaminolyticus, B. mojavensis, B. vallismortis, B. coagulans, B. sonorensis, B. halodurans, B. pocheonensis, B. gibsonii, B. acidiceler, B. flexus, B. hunanensis, B. pseudomycoides, B. simplex, B. safensis, B. mycoides, particularly preferred B. amyloliquefaciens, B. licheniformis, B. thuringiensis, B. velezensis, B. subtilis and B. megatherium, even more preferably B. amyloliquefaciens, B. thuringiensis, B. velezensis and B. megatherium. Clostridium species: C. autoethanogenum, C. beijerinckii, C. butyricum, C. carboxidivorans, C. disporicum, C. drakei, C. ljungdahlii, C. kluyveri, C. pasteurianum, C. propionicum, C. saccharobutylicum, C. saccharoperbutylacetonicum, C. scatologenes, C. tyrobutyricum, preferably C. butyricum, C. pasteurianum and/or C. tyrobutyricum,. C. aerotolerans, C. aminophilum, C. aminvalericum, C. celerecrescens, C. asparagforme, C. bolteae, C. clostridioforme, C. glycyrrhizinilyticum, C. (Hungatela) hathewayi, C. histolyticum, C. indolis, C. leptum, C. (Tyzzerella) nexile, C. perfringens, C. (Erysipelatoclostridium) ramosum, C. scindens, C. symbiosum, Clostridium saccharogumia, Clostridium sordelli, Clostridium clostridioforme, C. methylpentosum, C. islandicum and all members of the Clostridia clusters IV, XIVa, and XVIII, particularly preferred C. butyricum.

Some suitable Bacillus and Paenibacillus strains are described and deposited in the following international patent applications; spores of such microorganisms or pesticidally active variants of any thereof can be incorporated as spores of the composition according to the invention: WO2020200959: Bacillus subtilis or Bacillus amyloliquefaciens QST713 deposited under NRRL Accession No. B-21661 or a fungicidal mutant thereof. Bacillus subtilis QST713, its mutants, its supernatants, and its lipopeptide metabolites, and methods for their use to control plant pathogens and insects are fully described in U.S. Pat. Nos. 6,060,051, 6,103,228, 6,291,426, 6,417,163 and 6,638,910. In these patents, the strain is referred to as AQ713, which is synonymous with QST713; WO2020102592: Bacillus thuringiensis strains NRRL B-67685, NRRL B-67687, and NRRL B-67688; WO2019135972: Bacillus megatherium having the deposit accession number NRRL B-67533 or NRRL B-67534; WO2019035881: Paenibacillus sp. NRRL B-50972, Paenibacillus sp. NRRL B-67129, Bacillus subtilis strain QST30002 deposited under accession no. NRRL B-50421, and Bacillus subtilis strain NRRL B-50455; WO2018081543: Bacillus psychrosaccharolyticus strain deposited under ATCC accession number PT A-123720 or PT A-124246; WO2017151742: Bacillus subtilis assigned the accession number NRRL B-21661; WO2016106063: Bacillus pumilus NRLL B-30087; WO2013152353: Bacillus sp. deposited as CNMC 1-1582; WO2013016361: Bacillus sp. strain SGI-015-F03 deposited as NRRL B-50760, Bacillus sp. strain SGI-015-H06 deposited as NRRL B-50761; WO2020181053: Paenibacillus sp. NRRL B-67721, Paenibacillus sp. NRRL B-67723, Paenibacillus sp. NRRL B-67724, Paenibacillus sp. NRRL B-50374.

Most preferably the microorganism is a microorganism of taxonomic genus Paenibacillus and is selected from any of the species Paenibacillus polymyxa, Paenibacillus polymyxa polymyxa, Paenibacillus polymyxa plantarum and Paenibacillus terrae. Where the microorganism is a Paenibacillus microorganism, preferably the microorganism does not comprise both a A257V Spo0A mutation together with the Q218* DegU and/or L99F DegS mutation(s) as described according to the present invention. Even more preferably the microorganism is not Paenibacillus sp. strain NRRL B-67304, Paenibacillus sp. strain NRRL B-67306 or Paenibacillus sp. NRRL B-67615. The latter strains are described in WO2019221988. However, as described above this publication fails to observe the increased or stabilised exopolysaccharide production and/or reduced exopolysaccharide degradation as described in the present invention. All of these three strains are derived from Paenibacillus sp. strain NRRL B-67129, which, according to WO2016154297 examples 22, 25 and FIG. 16, contains the A257V mutation in the spoOA gene and further mutations. Further preferably the microorganism is not derived from Paenibacillus strain NRRL B-67129 deposited with the NRRL on 2015 Sep. 1. Thus, when aligning the genome of a Paenibacillus microorganism of the present invention—thereby excluding any extrachromosomal nucleic acids, for example plasmids—to the genome of (a) Paenibacillus strain NRRL B-67129 and (b) the type strain of the species of the Paenibacillus microorganism in question, the sequence identity of the Paenibacillus microorganism in question is preferably higher to the respective species type strain than to Paenibacillus strain NRRL B-67129. According to the invention, the following type strains are preferred:

| species | deposit |
|---|---|
| Paenibacillus abyssi | DSM 26238, CGMCC 1.12987 |
| Paenibacillus aestuarii | DSM 23861, JCM 15521, KACC 13125 |
| Paenibacillus agarexedens | DSM 1327, CIP 107437 |
| Paenibacillus agaridevorans | DSM 1355, CIP 107436 |
| Paenibacillus alginolyticus | DSM 5050, NRRL-NRS 1347 |
| Paenibacillus algorifonticola | DSM 26746, JCM 16598, XJ259, CGMCC 1.10223 |
| Paenibacillus alkaliterrae | DSM 17040, KCTC 3956, CIP 109150 |
| Paenibacillus alvei | DSM 29, ATCC 6344, CCM 2051, IFO 3343, IMAB B-3-4, LMG |
| | 13253, NCIB 9371, NCTC 6352, NBRC 3343 |
| Paenibacillus amylolyticus | DSM 15211, NRRL NRS-290 |
| Paenibacillus anaericanus | DSM 15890, ATCC BAA-844, CIP 109447, KCTC 13749 |
| Paenibacillus apiarius | DSM 5581, ATCC 29575, NRRL-NRS 1438, NCIMB 13506 |
| Paenibacillus arcticus | DSM 108811, KCTC 33985, JCM 30981, MME2_R6, PAMC 28731 |
| Paenibacillus azoreducens | DSM 13822, NCIMB 13761 |
| Paenibacillus azotifigens | DSM 104138, KACC 18967, LMG 29963 |
| Paenibacillus azotofixans | DSM 5976, ATCC 35681, LMG 14658 |
| Paenibacillus barcinonensis | DSM 15478, CECT 7022 |
| Paenibacillus barengoltzii | DSM 22255, NBRC 101215, ATCC BAA-1209, CIP 109354 |
| Paenibacillus beijingensis | DSM 24997, ACCC 03082 |
| Paenibacillus borealis | DSM 13188, CCUG 43137 |
| Paenibacillus bovis | DSM 28815, CGMCC 8333 |
| Paenibacillus brasilensis | DSM 14914, ATCC BAA-413, KCTC 3812, CIP 110686 |
| Paenibacillus brassicae | DSM 24983, ACCC 01125 |
| Paenibacillus campinasensis | DSM 21989, JCM 11200, KCTC 0364BP, BCRC 17341 |
| Paenibacillus castaneae | DSM 19417, CECT 7279 |
| Paenibacillus catalpae | DSM 24714, CGMCC 1.10784 |
| Paenibacillus cavernae | DSM 100100, KCTC 33652 |
| Paenibacillus cellulosilyticus | DSM 21372, CECT 5696, LMG 22232 |
| Paenibacillus chartarius | DSM 28439, CCUG 55240, CCM 7759 |
| Paenibacillus chibensis | DSM 11731, NRRL B-142, JCM 9905, IFO 15958, NBRC 15958 |
| Paenibacillus chinjuensis | DSM 15045, KCTC 8951, JCM 10939 |
| Paenibacillus chitinolyticus | DSM 11030, IFO 15660, NBRC 15660 |
| Paenibacillus chondroitinus | DSM 5051, NRRL-NRS 1351 |
| Paenibacillus chungangensis | DSM 28440, CCUG 59129, KCTC 13717 |
| Paenibacillus cineris | DSM 16945, CIP 108109, LMG 18439 |
| Paenibacillus cisolokensis | DSM 101873, UICC B-42, NRRL B-65368 |
| Paenibacillus cookii | DSM 16944, CIP 108110, LMG 18419 |
| Paenibacillus cucumis | DSM 101601, CCM 8655, LMG 29222 |
| Paenibacillus curdlanolyticus | DSM 10247, IFO 15724, NBRC 15724 |
| Paenibacillus daejeonensis | DSM 15491, KCTC 3745, JCM 11236 |
| Paenibacillus darwinianus | DSM 27245, ICMP 19912 |
| Paenibacillus dendritiformis | DSM 18844, CIP 105967 |
| Paenibacillus dongdonensis | DSM 27607, KCTC 33221 |
| Paenibacillus donghaensis | DSM 22278, LMG 23780, KCTC 13049 |
| Paenibacillus durus | DSM 1735, ATCC 27763, LMG 15707 |
| Paenibacillus ehimensis | DSM 11029, IFO 15659, NBRC 15659 |
| Paenibacillus elgii | DSM 22254, NBRC 100335, KCTC 10016BP, CIP 108552 |
| Paenibacillus elymi | DSM 106581, KCTC 33853 |
| Paenibacillus etheri | DSM 29760, CECT 8558 |
| Paenibacillus eucommiae | DSM 26048, KCTC 33054 |
| Paenibacillus faecis | DSM 23593, CIP 101062 |
| Paenibacillus favisporus | DSM 17253, LMG 20987, CECT 5760 |
| Paenibacillus filicis | DSM 23916, KCTC 13693, JCM 16417, KACC 14197 |
| Paenibacillus fonticola | DSM 21315, LMG 23577, BCRC 17579 |
| Paenibacillus forsythiae | DSM 17842, CCBAU 10203, CIP 110608 |
| Paenibacillus frigoriresistens | DSM 25554, JCM 18141, CCTCC AB 2011150 |
| Paenibacillus gansuensis | DSM 16968, KCTC 3950, CIP 109446 |
| Paenibacillus ginsengarvi | DSM 18677, KCTC 13059, CIP 109800 |
| Paenibacillus ginsengihumi | DSM 21568, JCM 14928, KCTC 13141 |
| Paenibacillus glucanolyticus | DSM 5162, ATCC 49278, IFO 15330, NCIB 12809, S93, NBRC 15330 |
| Paenibacillus glycanilyticus | DSM 17608, JCM 11221, CIP 107742, KCTC 3808, NRRL B-23455, DS-1 |
| Paenibacillus gorillae | DSM 26181, CSUR P205 |
| Paenibacillus graminis | DSM 15220, ATCC BAA-95, LMG 19080 |
| Paenibacillus harenae | DSM 16969, KCTC 3951 |
| Paenibacillus hodogayensis | DSM 22253, JCM 12520, KCTC 3919 |
| Paenibacillus hongkongensis | DSM 17642, CCUG 49571, CIP 107898 |
| Paenibacillus humicus | DSM 18784, NBRC 102415, LMG 23886, CIP 110609 |
| Paenibacillus hunanensis | DSM 22170, ACCC 10718, CGMCC 1.8907, CIP 110610 |
| Paenibacillus ihumii | DSM 100664, CSUR P1981, CSUR 1981 |
| Paenibacillus illinoisensis | DSM 11733, NRRL NRS-1356, JCM 9907, IFO 15959, NCIMB 13573, NBRC 15959 |
| Paenibacillus jilunlii | DSM 23019, CGMCC 1.10239, CIP110611 |
| Paenibacillus kobensis | DSM 10249, IFO 15729, NBRC 15729 |
| Paenibacillus konkukensis | DSM 104139, KACC 18876, LMG 29568 |
| Paenibacillus konsidensis | DSM 21992, JCM 14798, KCTC 13165 |
| Paenibacillus kribbensis | DSM 27933, JCM 11465, KCTC 0766BP |
| Paenibacillus lactis | DSM 15596, LMG 21940, CIP 108827 |
| Paenibacillus larvae | DSM 7030, ATCC 9545, LMG 9820, Med-540, NRRL B-2605 |
| Paenibacillus lautus | DSM 3035, ATCC 43898, LMG 11157, NCIMB 12780 |
| Paenibacillus lentus | DSM 25539, ATCC BAA-2594 |
| Paenibacillus macerans | DSM 24, ATCC 8244, CCM 2012, IAM 12467, LMG 13281, NCIB 9368, NCTC 6355, CIP 66.19 |
| Paenibacillus macquariensis subsp. | DSM 23149, JCM 14954, NCIMB 14397 |
| defensor | |
| Paenibacillus macquariensis subsp. | DSM 2, ATCC 23464, LMG 6935, CIP 103269, NCTC 10419 |
| macquariensis | |
| Paenibacillus marchantiophytorum | DSM 29850, CGMCC 1.15043 |
| Paenibacillus massiliensis subsp. | DSM 16942, CIP 107939, CCUG 48215 |
| massiliensis | |
| Paenibacillus mendelii | DSM 19248, CCM 4839, LMG 23002 |
| Paenibacillus motobuensis | DSM 18200, JCM 12774, CCUG 50090, GTC 1835 |
| Paenibacillus mucilaginosus | DSM 24461, CIP 105815, HSCC 1605, KCTC 3870, VKM B-1480D, VKPM B-7519 |
| Paenibacillus nanensis | DSM 22867, KCTC 13044, TISTR 1828, PCU 276 |
| Paenibacillus naphthalenovorans | DSM 14203, ATCC BAA-206 |
| Paenibacillus nebraskensis | DSM 103623, CIP 111179, LMG 29764 |
| Paenibacillus nematophilus | DSM 13559, CIP 108049 |
| Paenibacillus nicotianae | DSM 28018, NRRL B-59112, CGMCC 1.12819 |
| Paenibacillus odorifer | DSM 15391, ATCC BAA-93, LMG 19079 |
| Paenibacillus ottowii | DSM 107750, ATCC TSD-165 |
| Paenibacillus pabuli | DSM 3036, ATCC 43899, LMG 11158, NCIMB 12781 |
| Paenibacillus panacisoli | DSM 21345, KCTC 13020, LMG 23405 |
| Paenibacillus pasadenensis | DSM 19293, NBRC 101214, ATCC BAA-1211 |
| Paenibacillus pectinilyticus | DSM 28340, CECT 7358, KCTC 13222 |
| Paenibacillus peoriae | DSM 8320, LMG 14832, NRRL B-14750 |
| Paenibacillus phoenicis | DSM 27463, NBRC 106274, NRRL B-59348 |
| Paenibacillus phyllosphaerae | DSM 17399, LMG 22192, CECT 5862 |
| Paenibacillus physcomitrellae | DSM 29851, CGMCC 1.15044 |
| Paenibacillus pinihumi | DSM 23905, KCTC 13695, JCM 16419, KACC 14199 |
| Paenibacillus piri | DSM 105496, KACC 19385 |
| Paenibacillus pocheonensis | DSM 23906, KCTC 13941, LMG 23404 |
| Paenibacillus polymyxa | DSM 36, ATCC 842, BUCSAV 162, CCM 1459, JCM 2507, LMG 13294, NCIB 8158, NCTC 10343 |
| Paenibacillus popilliae | DSM 22700, CIP 106066, NRRL B-2309, ATCC 14706, CCUG 28881, NCCB 75017 |
| Paenibacillus prosopidis | DSM 22405, LMG 25259, CECT 7506 |
| Paenibacillus provencensis | DSM 22280, CCUG 53519, CIP 109358 |
| Paenibacillus pueri | DSM 22870, KCTC 13223, CECT 7360 |
| Paenibacillus puldeungensis | DSM 27603, KCTC 13718, CCUG 59189 |
| Paenibacillus purispatii | DSM 22991, CIP 110057 |
| Paenibacillus qingshengii | DSM 100926, JCM 30613, CCTCC AB 2014290 |
| Paenibacillus radicis | DSM 100762, CGMCC 1.15286 |
| Paenibacillus relictisesami | DSM 25385, JCM 18068 |
| Paenibacillus residui | DSM 22072, CCUG 57263 |
| Paenibacillus rhizophilus | DSM 103168, CGMCC 1.15699 |
| Paenibacillus rhizoplanae | DSM 103963, LMG 29875, CCM 8725 |
| Paenibacillus rhizosphaerae | DSM 17254, LMG 21955, CECT 5831 |
| Paenibacillus ripae | DSM 104672, LMG 29834, CCTCC AB 2014276, LMG 28639 |
| Paenibacillus sabinae | DSM 17841, CCBAU 10202, CIP 109632, KCTC 13697 |
| Paenibacillus sacheonensis | DSM 23054, KACC 14895, CIP 110612 |
| Paenibacillus sanguinis | DSM 16941, CIP 107938, CCUG 48214 |
| Paenibacillus sediminis | DSM 23491, GT-H3, LMG 25635, CIP 110613 |
| Paenibacillus segetis | DSM 28014, CGMCC 1.12769 |
| Paenibacillus senegalensis | DSM 25958, CSUR P157 |
| Paenibacillus sepulcri | DSM 27042, LMG 19508, CCM 7311, Heyrman R-514, mcha6024 |
| Paenibacillus shirakamiensis | DSM 26806, NBRC 109471, KCTC 33126, CIP 110571 |
| Paenibacillus silagei | DSM 101953, JCM 30974 |
| Paenibacillus silvae | DSM 28013, CGMCC 1.12770 |
| Paenibacillus solani | DSM 100999, CCTCC AB 2015207 |
| Paenibacillus soli | DSM 21316, LMG 23604, KCTC 13010 |
| Paenibacillus sonchi | DSM 28159, CECT 7330, CCGB 1313 |
| Paenibacillus sophorae | DSM 23020, CGMCC 1.10238, CIP 110614 |
| Paenibacillus sputi | DSM 22699, KCTC 13252 |
| Paenibacillus stellifer | DSM 14472 |
| Paenibacillus taichungensis | DSM 19942, BCRC 17757, CIP 110615 |
| Paenibacillus taiwanensis | DSM 18679, IAM 15414, LMG 23799 |
| Paenibacillus taohuashanense | DSM 25809, CGMCC 1.12175 |
| Paenibacillus tarimensis | DSM 19409, CCTCC AB 206108, CIP 110616 |
| Paenibacillus terrae | JCM: 11466, KCCM: 41557 |
| Paenibacillus terreus | DSM 100035, KACC 18491, CCTCC AB 2015273 |
| Paenibacillus terrigena | DSM 21567, CCTCC AB206026, IAM 15291, JCM 21741 |
| Paenibacillus thailandensis | DSM 22866, KCTC 13043, TISTR 1827, PCU 275 |
| Paenibacillus thermoaerophilus | DSM 26310, JCM 18657 |
| Paenibacillus thiaminolyticus | DSM 7262, JCM 8360, NRRL B-4156 |
| Paenibacillus tianmuensis | DSM 22342, CGMCC 1.8946, CIP 110617 |
| Paenibacillus tibetensis | DSM 29321, ACCC 19728 |
| Paenibacillus timonensis | DSM 16943, CIP 108005, CCUG 48216 |
| Paenibacillus triticisoli | DSM 25425, CGMCC 1.12045 |
| Paenibacillus tundrae | DSM 21291, NRRL B-51094, CIP 110036 |
| Paenibacillus turicensis | DSM 14349, NCCB 100011 |
| Paenibacillus tylopili | DSM 18927, LMG 23975 |
| Paenibacillus typhae | DSM 25190, CGMCC 1.11012, CIP 110618 |
| Paenibacillus uliginis | DSM 21861, LMG 24790 |
| Paenibacillus urinalis | DSM 22281, CCUG 53521, CIP 109357 |
| Paenibacillus validus | DSM 3037, ATCC 43897, LMG 11161, NCIMB 12782, NRRL-NRS 1000 |
| Paenibacillus vulneris | DSM 27954, JCM 18268, CCUG 53270 |
| Paenibacillus wenxiniae | DSM 100576, CGMCC 1.15007 |
| Paenibacillus woosongensis | DSM 16971, KCTC 3953, CIP 110595 |
| Paenibacillus wulumuqiensis | DSM 29194, CPCC 100602, JCM 30284 |
| Paenibacillus wynnii | DSM 18334, CIP 108306, LMG 22176 |
| Paenibacillus xerothermodurans | DSM 520, ATCC 27380 |
| Paenibacillus xinjiangensis | DSM 16970, KCTC 3952, CIP 109466 |
| Paenibacillus xylanexedens | DSM 21292, NRRL B-51090, CIP 110619 |
| Paenibacillus xylaniclasticus | DSM 26531, NBRC 106381, KCTC 13719, TISTR 1914 |
| Paenibacillus xylanilyticus | DSM 17255, LMG 21957, CECT 5839 |
| Paenibacillus xylanisolvens | DSM 25299, KCTC 13042, PCU 311, TISTR 1829 |
| Paenibacillus xylanivorans | DSM 107920, NCIMB 15123 |
| Paenibacillus zanthoxyli | DSM 18202, CCBAU 10243, CIP 109595, KCTC 13696 |

If, for a microorganism in question, the species cannot be reliably decided, then it is sufficient that the genome of the microorganism in question has a greater sequence identity to the genome of any of the aforementioned preferred type strains than to the genome of Paenibacillus strain NRRL B-67129.

The invention also provides a method of increasing or stabilising exopolysaccharide production or of reduction or prevention of exopolysaccharide degradation of a microorganism, comprising the step of providing, in the microorganism one or more of
a) a mutant DegU protein as described herein according to the invention,
b) a mutant DegS protein as described herein according to the invention,
c) a mutant Spo0A protein as described herein according to the invention.

As described herein, providing such mutant protein or mutant proteins allows to achieve the advantages offered by the present invention, in particular a reduction of exopolysaccharide degradation in late fermentation stages and/or a stabilisation of maximum fermentation broth viscosity. Thus, where the microorganism furthermore produces a substance of interest, for example one or more fusaricidins the present invention advantageously allows to harvest the fermentation broth when both the highest viscosity/exopolysaccharide content of the one hand and the highest concentration of the substance of interest is achieved, thereby obviating the need to decide between either a maximum concentration of the substance of interest or a maximum exopolysaccharide content.

Correspondingly the invention also provides a method for microbial exopolysaccharide production, comprising the steps of
i) fermenting a microorganism of the present invention to produce a fermentation broth containing one or more exopolysaccharides and preferably one or more target fermentation products, and
ii) enriching the one or more exopolysaccharides, and preferably also the one or more target fermentation products, from said fermentation broth.

As described herein, it is a particular advantage of the microorganisms of the present invention comprising the alleles of the degU, degS and/or spoOA genes, as applicable, that these microorganisms are capable of increased or stabilised exopolysaccharide production and/or reduced exopolysaccharide degradation. Thus, the invention advantageously allows to harvest a fermentation at a point where both a high content of exopolysaccharides and a high content of target fermentation products can be harvested. Using the teaching of the present invention, the harvesting time can be prolonged until sufficient target fermentation product yield is reached without having to suffer losses of exopolysaccharide yield as would occur if an exopolysaccharide-degrading microorganism had been used.

Techniques for increasing exopolysaccharde production in fermentations and/or for enriching of exopolysaccharides are known to the skilled person. They are described, for example, in any of: Liang et al., Recent Advances in Exopolysaccharides from Paenibacillus spp.: Production, Isolation, Structure, and Bioactivities, Mar. Drugs 2015, 13, 1847-1863, doi: 10.3390/md13041847; Sun et al., Extraction of extracellular polymeric substances in activated sludge using sequential extraction, J Chem Technol Biotechnol 2015; 90:1448-1454, DOI 10.1002/jctb.4449; Chen Xu, Optimised Procedures for Extraction, Purification and Characterization of Exopolymeric Substances (EPS) from Two Bacteria With Relevance to the Study of Actinide Binding in Aquatic environments; Ms.Science Thesis, December 2007, Texas A&M University. Enrichment typically involves pecipitation or cation exchange resin extraction of the fermentation broth supernatant to obtain a crude exopolysaccharide fraction. The crude exopolysaccharide fraction can be further purified, e.g. by chromatography.

The invention also provides an expression vector comprising an expression cassette for expression of one or more of
a) a mutant DegU protein as described herein according to the invention,
b) a mutant DegS protein as described herein according to the invention,
c) a mutant Spo0A protein as described herein according to the invention.

Such expression vector allows to introduce the respective mutant gene or genes into a wildtype organism, either in addition to or as a replacement for the respective wild type gene. Thus, the expression vector of the present invention allows for a particularly easy conversion of a wild-type microorganism into a microorganism of the present invention.

As indicated above, the microorganism according to the present invention preferably has agronomic relevance. Correspondingly it is a particular advantage that the present invention provides a method of plant health improvement, wherein the method comprises the application of a microorganism of the present invention to
a) plant material and/or
b) a plant cultivation substrate.

The increase and/or stabilisation of exopolysaccharide production and/or reduction of exopolysaccharide degradation of the microorganism of the present invention advantageously allows for an increase of the residence time of the microorganism at the location it is originally applied to. The probiotic microorganism according to the present invention is capable of increased and/or stabilised exopolysaccharide production and/or reduced exopolysaccharide degradation; thus, the microorganism will tend to be embedded in a larger exopolysaccharide coat compared to the corresponding wild-type microorganism. The exopolysaccharides, in turn, prevent a dislocation of the microorganism of the present invention and, for example, protect the microorganism against being rinsed off by rainfall or being blown away by wind.

Preferably the microorganism of the present invention produces, during a fermentation or after application to a plant material or a plant cultivation substance, a plant beneficial compound. Thus, the microorganism of the present invention preferably is a plant probiotic microorganism.

The plant health promotion composition of the present invention is preferably applied to the plant material, preferably a plant propagation material, by any step of dressing, spraying, coating, film coating, pelleting, dusting or soaking.

The plant probiotic microorganism of the present invention preferably is applied to a plant surface, preferably a plant leaf. Because of the improved exopolysaccharide production and/or reduced exopolysaccharide degradation, the probiotic microorganism of the present invention will show an increase in immobility at the site of application. This is particularly advantageous to provide an anti-pathogenic coating on the plant surface, most preferably a leaf surface. Most preferably, the probiotic microorganism of the present invention colonises the plant surface, thereby further extending its beneficial, preferably anti-pathogenic effects to the plant. In this context, microorganisms of the present invention of genus Paenibacillus are particularly preferred in view of the protection they offer to the plant against fungal infections.

In addition, or as an alternative to application of the probiotic microorganism of the present invention to a plant surface, the probiotic microorganism of the present invention is preferably applied to a plant cultivation substrate, most preferably soil. Again, due to the improved exopolysaccharide production and/or reduced exopolysaccharide degradation of the microorganism of the present invention, said microorganism is retained at the site of application for a longer time compared to the respective wild type microorganism. Thus, most preferably the probiotic microorganism of the present invention colonises the plant cultivation substrate at the site of application, thereby improving the quality of the plant growth substrate and making it more amenable to improved plant growth.

Thus, the invention allows to improve soil fertility and/or improvement of yield consistency, thereby further advancing the teachings of each of WO2015118516, WO2016044768 and WO2020163251, each incorporated herein by reference.

Preferably the plant beneficial compound is a target fermentation product as described herein. Such compounds and mixtures thereof have antimicrobial, preferably anti-fungal properties. Thus, the microorganism of the present invention advantageously improves plant health and/or yield and/or yield consistency when applied to a plant cultivation substrate and/or a plant material as described herein.

In particular, the microorganism of the present invention is preferably used to prevent, delay and/or reduce infections of plant materials, preferably of whole plants, by a microorganism plant pathogen, preferably a fungus. Preferably the microorganism of the present invention is used to prevent, delay and/or reduce infections of plant materials, preferably of whole plants, by a microorganism selected from any of:
class Gammaproteobacteria, more preferably order Xanthomonadales, more preferably of family Xanthomonadaceae, more preferably of genus Xanthomonas;
class Sordariomycetes, more preferably of order Hypocreales, more preferably of family Nectriaceae, more preferably of genus Fusarium;
class Sordariomycetes, more preferably of order Glomerellales, more preferably of family Glomerellaceae, more preferably of genus Colletotrichum;
class Leotinomycetes, more preferably of order Helotiales, more preferably of family Sclerotiniaceae, more preferably of genus Botrytis;
class Dothideomycetes, more preferably of order Pleosporales, more preferably of family Pleosporaceae, more preferably of genus Alternaria;
class Dothideomycetes, more preferably of order Pleosporales, more preferably of family Phaeosphaeriaceae, more preferably of genus Phaeosphaeria;
class Dothideomycetes, more preferably of order Botryosphaeriales, more preferably of family Botryosphaeriaceae, more preferably of genus Macrophomina;
class Dothideomycetes, more preferably of order Capnodiales, more preferably of family Mycosphaerellaceae, more preferably of genus Zymoseptoria;
class Agraricomycetes, more preferably of order Cantharellales, more preferably of family Ceratobasidiaceae, more preferably of genus Rhizoctonia or Thanatephorus;
class Pucciniomycetes, more preferably of order Pucciniales, more preferably of family Pucciniaceae, more preferably of genus Uromyces or Puccinia;
class Ustilaginomycetes, more preferably of order Ustilaginales, more preferably of family Ustilaginaceae, more preferably of genus Ustilago;
class Oomycota, more preferably of order Pythiales, more preferably of family Pythiaceae, more preferably of genus Pythium;
class Oomycota, more preferably of order Peronosporales, more preferably of family Peronosporaceae, more preferably of genus Phytophthora, Plasmopara or Pseudoperonospora.
class Eurotiomycetes, more preferably of order Eurotiales or Onygenales, more preferably of family Aspergillaceae, more preferably of genus Aspergillus, Penicillium or Pseudopenicillium; even more preferably genus Alternaria, Botrytis, Fusarium, Sclerotinia or Trichoderma.

The microorganism of the present invention can be applied in the form of active cells (that is, non-sporulated cells that have an active metabolism and can divide) to the plant material and/or plant cultivation substance. As described herein, the microorganism can colonise the plant material and/or cultivation substrate to exert its beneficial properties. However, preferably the microorganism is applied in the form of spores of said microorganism, optionally together with active cells of the microorganism. Spores allow the microorganism to withstand conditions unsuitable for growth and survival of active cells. In particular, harvesting of fermenters, downstream processing, storage and high pressure spraying are steps generally used to manufacture a plant health product, but each of these steps can cause a significant reduction of the content of surviving active cells. Spores, on the other hand, can easily survive these conditions and are thus particularly suitable for the manufacture of a plant health product.

The invention correspondingly provides a plant health product comprising a microorganism culture (preferably comprising spores and/or active cells) of one or more microorganisms of the present invention. The microorganism can be comprised in the plant health product in the form of a mixed culture consisting of different species of microorganisms and/or different strains of a species of microorganisms. Alternatively, the microorganism culture preferably is a pure culture consisting of one species of one microorganism and even more preferably consists of one strain of one species of a microorganism of the present invention.

When at least one microorganism of the microorganism culture produces spores, then preferably such spores are harvested. Harvesting techniques like centrifugation, filtration and gear filtration are known to the person skilled in the art. It is a particular advantage of the fermentation method of the present invention that high titers of spores with a high content of antifungal substances, notably fusaricidins, can be produced with low efforts in a conveniently short time and with high antifungal activity.

It is also preferred to harvest a cell free suspension at the end of the fermentation method of the present invention. Again, techniques for obtaining a cell free suspension unknown to the person skilled in the art can favourably be combined with methods for harvesting spores.

The invention also provides a plant health promotion composition, obtainable or obtained by a method according to the present invention. As described herein, such compositions are surprisingly effective, and they are easy and fast and cost effectively to produce.

The plant health composition optionally further comprises a stabilizing agent, preferably as disclosed in WO2019222253A, and also preferably one or more target fermentation products as described above. Furthermore, the plant health composition of the present invention preferably further comprises
a) one or more microbial pesticides with fungicidal, bactericidal, viricidal and/or plant defense activator activity,
b) one or more biochemical pesticides with fungicidal, bactericidal, viricidal and/or plant defense activator activity,
c) one or more microbial pesticides with insecticidal, acaricidal, molluscidal and/or nematicidal activity,
d) one or more biochemical pesticides with insecticidal, acaricidal, molluscidal, pheromone and/or nematicidal activity,
e) one or more fungicide selected from respiration inhibitors, sterol biosynthesis inhibitors, nucleic acid synthesis inhibitors, inhibitors of cell division and cytoskeleton formation or function, inhibitors of amino acid and protein synthesis, signal transduction inhibitors, lipid and membrane synthesis inhibitors, inhibitors with multi-site action, cell wall synthesis inhibitors, plant defence inducers and fungicides with unknown mode of action.

The further components a)-d) are described in WO2017137353, which is incorporated herein for the purpose of enumerating the respective substances. The further components e) are described in WO2017137351, which is also incorporated herein for the purpose of enumerating the respective fungicides.

The invention also provides a method of exopolysaccharide production, comprising growing a microorganism according to the invention and, optionally, separating the microorganism from the exopolysaccharide. Suitable methods of growing a microorganism of the present invention, that is, fermentation methods, are generally known to the person skilled in the art. It is a particular advantage that the improved yield in exopolysaccharides can be achieved according to the invention without fundamental changes in corresponding fermentation processes.

In extension of the advantages of the present invention, further provided is a use of a microorganism according to the invention or a mutant DegU protein or gene and/or a mutant DegS protein or gene and/or a mutant Spo0A protein or gene for any of:
production of an exopolysaccharide composition,
treatment of plants, plant leaves, plant roots and/or plant seed,
inoculation of soil, preferably for enhancing soil fertility,
improvement of yield consistency,
treatment of subterraneous formations,
treatment of wastewater,
preparation of a pharmaceutical or cosmetic carrier,
preparation of a pharmaceutical or cosmetic composition,
preparation of a skin hydration composition,
preparation of a flocculant,
preparation of a food or feed additive,
preparation of an antitumor agent
preparation of an antioxidant.

Particularly suitable methods of using exopolysaccharides and/or microorganisms for the treatment of subterraneous formations are described in WO2014176061. Particularly suitable methods of using exopolysaccharides and/or microorganisms for the treatment of wastewater are described in WO2014160350, both incorporated herein by reference.

The invention also provides the use of one or more of
a) a mutant DegU protein according to the present invention,
b) a mutant DegS protein according to the present invention,
c) a mutant Spo0A protein according to the present invention,
   for increasing or stabilising of exopolysaccharide production or prevention of exopolysaccharide degradation of a microorganism selected from any of the taxonomic ranks defined above.

Selected aspects of the inventiion are hereinafter further described by means of the following, non-limiting examples.

### EXAMPLES

### Example 1: Mutant Generation

### Strains and Cultivation Conditions

A list of strains used for targeted integration of point mutations by CRISPR Cas9 in P. polymyxa is shown in table 1. Targeted point mutations in wildtype strain P. polymyxa DSM365 were integrated according to the CRISPR Cas9 procedure described in Rütering et. al (Rütering et al., Tailor-made exopolysaccharides-CRISPR-Cas9 mediated genome editing in Paenibacillus polymyxa. Synth Biol (Oxf). 2017 Dec. 21; 2 (1): ysx007. doi: 10.1093/synbio/ysx007). DSM 365 was obtained from the German Collection of Microorganisms and Cell Culture (DSMZ), Braunschweig, Germany. Plasmid cloning and multiplication were performed in either E. coli DH5a or Turbo from NEB (New England Biolabs, USA). Transformation of P. polymyxa was performed by conjugation mediated by E. coli S17-1 (DSMZ). The strains were grown in LB media (10 g/L tryptone peptone, 5 g/L yeast extract, 5 g/L NaCl). For plate media, 1.5% agar was used. Whenever necessary, the media was supplemented with 50 μg/ml neomycin and/or 20 μg/mL polymyxin for counterselection of positive transformants and to get rid of E. coli after the conjugation procedure. P. polymyxa was grown at 30° C. and 250 rpm while E. coli at 37° C. and 250 rpm, unless stated otherwise. The strains were stored as cryo culture with 24% glycerol and kept at −80 C for longer storage.

TABLE 1
List of strains used for CRISPR Cas9 mediated construction of targeted point-mutations in P. polymyxa DSM365

| Strain | Description | Reference | Use |
|---|---|---|---|
| E. coli DH5α | F− ϕ80lacZΔM15 Δ (lacZYA-argF) | NEB | High copy plasmid |
| | 169 recA1 endA1 hsd R17 (rK−, K+) | | amplification of pCasPP |
| | phoA supE44 λ− thi-1 gyrA96 relA1 | | before transformation |
| | | | in S17-1 |
| E. coli Turbo | F′ proA + B+ laclq ΔlacZM15/fhuA2 Δ(lac- | NEB | High copy plasmid |
| | proAB) glnV galK16 galE15 R(zgb- | | amplification of pCasPP |
| | 210::Tn10)TetS endA1 thi-1 Δ(hsdS-mcrB)5 | | before transformation |
| | | | in S17-1 |
| E. coli S17-1 | recA pro hsdR RP42Tc::Mu-Km::Tn7 integrated | ATCC 47055 | Conjugation strain: donor |
| | into the chromosome | (DSM 9079) | |
| P. polymyxa DSM365 | Wild type (public strain) | DSM365/BASF # | Target strain for modification |
| | | LU21039 | |

### Conjugation

Conjugation was performed between P. polymyxa (recipient strain) and E. coli S17-1 harboring the plasmid of interest (donor strain) according to the CRISPR Cas9 procedure described in Rütering et al. 2017 (Rutering M, Cress B F, Schilling M, Rühmann B, Koffas M A G, Sieber V, Schmid J. Tailor-made exopolysaccharides-CRISPR-Cas9 mediated genome editing in Paenibacillus polymyxa. Synth Biol (Oxf). 2017 Dec. 21; 2 (1): ysx007. doi: 10.1093/synbio/ysx007. PMID: 32995508; PMCID: PMC7445874). Confirmation of the correct conjugants was performed by colony PCR and sequencing of DNA fragments. Plasmid curing was performed by 1:100 subculturing of the positive mutant in LB liquid media at 37° C.

### Plasmid Construction

Targeted point mutations were achieved by CRISPR-Cas9 mediated system. Selected gRNA sequences were chosen based on their closest proximity to the targeted positions within degU, degS, or spo0A genes. The plasmids were assembled by isothermal Gibson Assembly. Desired point mutations were introduced from the primers used for PCR of the homology flanks. For degS and spoOA, several silent mutations were also introduced in the primers to improve efficiency of the system. Homology flanks were obtained by PCR of P. polymyxa genomic DNA, about 1 kbp upstream and downstream of the targeted nucleotides. E. coli DH5α or Turbo was transformed with the Gibson assembly mixture and plated on LB plate containing 50 μg/ml neomycin. Screening of the positive colonies was done by colony PCR. Plasmids were isolated by miniprep and verified by sequencing for further confirmation. The correct plasmid was used to transform E. coli S17-1 which would then mediate the transformation to P. polymyxa.

Using the pCasPP vector system and homologues flanks carrying 1000 bp, each, of the surrounding genomic sequences flanking the targeted point mutation region, the following mutations were generated (table 2):

TABLE 2
List of mutant strains and associated spacer
sequences used for genome editing by CRISPR Cas9.

| | | Modification | spacer sequence |
|---|---|---|---|
| strain | gene | (Amino acid / nucleotide position) | 5′ -> 3′ |
| DSM365 | degU | SNP: | CAGCAGTATTTTGCAAAAAA |
| | | degU Q218* (nt. pos. 652 C → T) | |
| DSM365 | degU | Insertion: | CAGCAGTATTTTGCAAAAAA |
| | | degU D223* + M220N + E221G + V222G | |
| | | (Insertion at nt. pos. 658 A → AA | |
| DSM365 | degS | SNP: | TGTGATGATTTTCCGCGAGA |
| | | degS L99F (nt. pos. 295 C → T) | |
| DSM365 | spoOA | SNP: | TAAGCTGAGAATTGAGAACA |
| | | spo0A A257V (nt. pos. 770 C → T) | |
| DSM365 | degS + | Double mutant | See above |
| | degU | degS L99F + degU Q218* | |
| DSM365 | spoOA + | Triple mutant | See above |
| | degS + | spo0A A257V + degS L99F + degU Q218* | |
| | degU | | |

SNP = single nucleotide polymorphism, nt = nucleotide.

### Example 2: Fermentation Conditions

Characterization of mutants was carried out in 211 bioreactors (Techfors, Infors), filled with 121 exopolysaccharide production medium adapted from Rütering et. al 2017. The composition of the fermentation medium is listed in table 3.

TABLE 3
Composition of the exopolysaccharide production medium
with the specification for storage (room temperature
(RT) or 4° C.) and sterilization method (sterile-
filtered/autoclaved, s/a) of the stock solution.

| | | Concentration in |
|---|---|---|
| Stock solution | Component | the medium [g/l] |
| Main solution | Potassium dihydrogen phosphate | 1.67 |
| (RT, a) | Magnesium sulfate heptahydrate | 1.33 |
| | Calcium chloride dihydrate | 0.05 |
| | Peptone from soy | 5.0 |
| Sugar (RT, a) | Glucose monohydrate | 30 |
| Vitamin solution | Thiamin hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxin hydrochloride | 0.005 |
| | Vitamin B12 | 0.00005 |
| | Lipoic acid | 0.00005 |
| Trace element | Manganese sulfate monohydrate | 0.013 |
| solution | Copper sulfate pentahydrate | 0.0046 |
| (4° C., s) | Sodium molybdate dihydrate | 0.0028 |
| | Iron sulfate monohydrate | 0.015 |
| | Citric acid monohydrate | 0.4 |

Fermentation took place at 30° C. for 40 h, pH was set to 6.8 and adjusted with H3PO4 (25%) and NaOH (1M). As preculture, all mutants were grown for 24 h in 1 L shake flasks with baffles containing 100 ml of modified TSB medium (30 g/L TSB from Becton Dickenson Art.Nr.211825, 3 g/L yeast extract, 20.9 g/L MOPS buffer, 10 g/L glucose) at 33° C. and 150 rpm/2.5 cm throw.

In the bioreactor, target dissolved oxygen level was set a ≥30% in a stirrer-gas flow cascade. To prevent sheering of the exopolysaccharides produced, agitation was limited to 300-600 rpm while using a stirrer setup consisting of two propellers and one Rushton, the latter was placed near the agitator shaft. To maintain oxygen supply, aeration was performed at 5-30 l/min at 0.5 bar pressure. Struktol J673 (Schill+Seilacher “Struktol” GmbH, Germany) was used as antifoam agent. Culture samples were taken every 4h for rheological viscosity analyses and further offline analytics.

### Example 3: Rheological Analyses of Culture Broth Viscosity

Rheological analysis of broth viscosity was conducted every 4h during the fermentation using an Anton Paar MCR302 rheometer with double slid geometry (Measuring Cup: C-DG26.7/SS/Air, temperature: 30° C., sample volume: 5 ml of whole culture broth). The samples were preconditioned in a pre-shear experiment at constant shear rate of 10 s−1 for 100 s. 10 data points were recorded every 10 s. After preconditioning, viscosity was measured as a function of the shear rate. Therefore, the shear rate was logarithmically increased from 1 s−1 to 100 s−1 while logging a total of 25 data points. Culture broth viscosity measured over the course of time of the fermentation is depicted in FIG. 1.

### Example 4: Determination of Carbon Transfer Rate

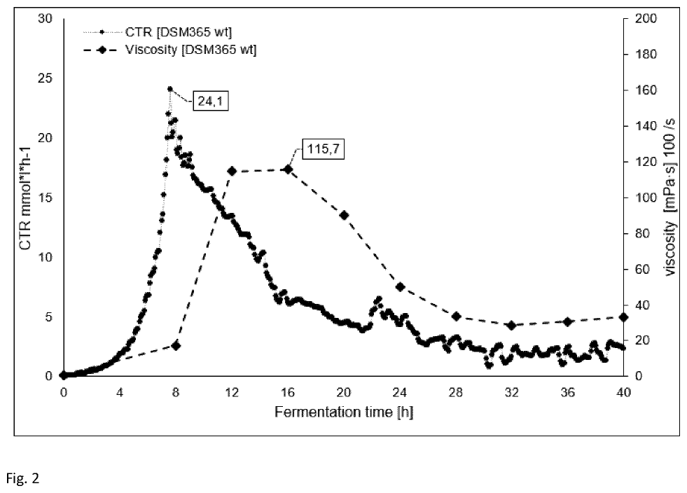

Carbon transfer rate (CTR, in mmol/l*h-1) was assessed according to the protocol of Anderlei et al. (Anderlei, Tibor & Zang, Werner & Papaspyrou, Manfred & Buchs, Jochen. (2004). Online respiration activity measurement (OTR, CTR, RQ) in shake flasks. Biochemical Engineering Journal. 17. 187-194. 10.1016/S1369-703X (03) 00181-5) every 5 minutes in the headspace of the 21L fermenters from example 2 using a mass spectrometer. The carbon transfer rate was used as an online indicator for the metabolic activity of the strains. The profile of the carbon transfer rate and viscosity of Paenibacillus DSM365 is exemplarily shown in FIG. 2. The maximum viscosity of the fermentation broth is reached after the maximum CTR. This behaviour is found also for the degU, degS and spoOA mutant strains discussed in FIG. 1 (data not shown).

1. A microorganism comprising a mutant degU gene and/or a mutant degS gene, and optionally further a mutant spo0A gene,
wherein the microorganism exhibits increased and/or stabilised exopolysaccharide production and/or reduced exopolysaccharide degradation.

2. The microorganism according to claim 1, comprising a mutant degU gene, wherein
the degU gene codes for a DegU protein having reduced DNA binding activity and/or lacks a functional DNA binding domain, and/or
the degU gene codes for a DegU protein, wherein the mutation comprises or consists of one or more of:
a) Q218*, Q218K, Q218N, Q218D, or Q218R, and/or
b) D223*, D223*+M220N, D223*+M220N+E221G, D223*+M220N+V222G, D223*+M220N+E221G+V222G, D223*+M220D, D223*+M220E, D223*+M220H, D223*+M220F, D223*+M220W, D223*+M220S, or D223*+M220A.

3. The microorganism according to claim 1, comprising a mutant degS gene, wherein
the degS gene codes for a DegS protein lacking a functional single binding domain, a functional phospoacceptor domain and/or a functional ATPase domain and/or
the degS gene codes for a DegS protein, wherein the mutation comprises or consists of L99F, L99C, L99D, L99E, L99G, L99H, L99K, L99N, L99P, L99Q, L99R, L99S, L99W or L99Y.

4. The microorganism according to claim 1, comprising a mutant spo0A gene, wherein
a) the mutation is located in the DNA binding or receiver domain and results in a reduction or elimination of phosphorylation of the Spo0A protein and/or a reduction or elimination of dimerisation, and/or
b) the mutation consists of or comprises any of
A257V, or
I161R, or
A257S+I161I, A257A+I161L, A257V+1161I, A257S+I161F or A257A+I161R.

5. The microorganism according to claim 1, wherein the microorganism, when grown in a liquid fermentation medium, causes a viscosity increase of the fermentation medium such that the fermentation medium viscosity remains higher than 50% of the maximal fermentation medium viscosity obtained in a fermentation of the corresponding wild type strain.

6. The microorganism according to claim 1, wherein the microorganism is selected from a taxonomic rank selected from the group consisting of
phylum Firmicutes, class Bacilli, Clostridia or Negativicutes,
order Bacillales, Clostridiales, Thermoanaerobacterales, Thermosediminibacterales or Selenomonadales,
family Bacillaceae, Paenibacillaceae, Pasteuriaceae, Clostridiaceae, Peptococcaceae, Heliobacteriaceae, Syntrophomonadaceae, Thermoanaerobacteraceae, Tepidanaerobacteraceae or Sporomusaceae,
genus Alkalibacillus, Bacillus, Geobacillus, Halobacillus, Lysinibacillus, Piscibacillus, Terribacillus, Brevibacillus, Paenibacillus, Thermobacillus, Pasteuria, Clostridium, Desulfotomaculum, Heliobacterium, Pelospora, Pelotomaculum, Caldanaerobacter, Moorella, Thermoanaerobacter, Tepidanaerobacter, Propionispora or Sporomusa, and
genus Bacillus, Paenibacillus or Clostridium.

7. A method of increasing or stabilising exopolysaccharide production or of reduction or prevention of exopolysaccharide degradation of a microorganism, comprising the step of providing, in the microorganism, the mutant DegU protein according to claim 2.

8. An expression vector, comprising an expression cassette for expression of the mutant DegU protein according to claim 2.

9. A method of plant health improvement, comprising applying the microorganism according to claim 1, to
a) plant material and/or
b) a plant cultivation substrate.

10. A method of exopolysaccharide production, comprising
i) growing the microorganism according to claim 1, and
ii) optionally separating the microorganism from the exopolysaccharide.

11. A method of using the microorganism according to claim 1, the method comprising using the microorganism for an application selected from the group consisting of:
production of an exopolysaccharide composition,
treatment of plants, plant leaves, plant roots and/or plant seed,
inoculation of soil, preferably for enhancing soil fertility,
improvement of yield consistency,
treatment of subterraneous formations,
treatment of wastewater,
preparation of a pharmaceutical or cosmetic carrier,
preparation of a pharmaceutical or cosmetic composition,
preparation of a skin hydration composition,
preparation of a flocculant,
preparation of a food or feed additive
preparation of an antitumor agent, and
preparation of an antioxidant.

12. A method of using the mutant DegU protein according to claim 2, the method comprising using the mutant DegU protein for increasing or stabilising of exopolysaccharide production or prevention of exopolysaccharide degradation of a microorganism selected from a taxonomic rank selected from the group consisting of
phylum Firmicutes, class Bacilli, Clostridia or Negativicutes,
order Bacillales, Clostridiales, Thermoanaerobacterales, Thermosediminibacterales or Selenomonadales,
family Bacillaceae, Paenibacillaceae, Pasteuriaceae, Clostridiaceae, Peptococcaceae, Heliobacteriaceae, Syntrophomonadaceae, Thermoanaerobacteraceae, Tepidanaerobacteraceae or Sporomusaceae,
genus Alkalibacillus, Bacillus, Geobacillus, Halobacillus, Lysinibacillus, Piscibacillus, Terribacillus, Brevibacillus, Paenibacillus, Thermobacillus, Pasteuria, Clostridium, Desulfotomaculum, Heliobacterium, Pelospora, Pelotomaculum, Caldanaerobacter, Moorella, Thermoanaerobacter, Tepidanaerobacter, Propionispora or Sporomusa, and
genus Bacillus, Paenibacillus or Clostridium.

13. A method of using the mutant DegS protein according to claim 3, the method comprising using the mutant DegS protein for increasing or stabilising of exopolysaccharide production or prevention of exopolysaccharide degradation of a microorganism selected from a taxonomic rank selected from the group consisting of
phylum Firmicutes, class Bacilli, Clostridia or Negativicutes,
order Bacillales, Clostridiales, Thermoanaerobacterales, Thermosediminibacterales or Selenomonadales,
family Bacillaceae, Paenibacillaceae, Pasteuriaceae, Clostridiaceae, Peptococcaceae, Heliobacteriaceae, Syntrophomonadaceae, Thermoanaerobacteraceae, Tepidanaerobacteraceae or Sporomusaceae,
genus Alkalibacillus, Bacillus, Geobacillus, Halobacillus, Lysinibacillus, Piscibacillus, Terribacillus, Brevibacillus, Paenibacillus, Thermobacillus, Pasteuria, Clostridium, Desulfotomaculum, Heliobacterium, Pelospora, Pelotomaculum, Caldanaerobacter, Moorella, Thermoanaerobacter, Tepidanaerobacter, Propionispora or Sporomusa, and
genus Bacillus, Paenibacillus or Clostridium.

14. A method of using the mutant Spo0A protein according to claim 4, the method comprising using the mutant Spo0A protein for increasing or stabilising of exopolysaccharide production or prevention of exopolysaccharide degradation of a microorganism selected from a taxonomic rank selected from the group consisting of
phylum Firmicutes, class Bacilli, Clostridia or Negativicutes,
order Bacillales, Clostridiales, Thermoanaerobacterales, Thermosediminibacterales or Selenomonadales,
family Bacillaceae, Paenibacillaceae, Pasteuriaceae, Clostridiaceae, Peptococcaceae, Heliobacteriaceae, Syntrophomonadaceae, Thermoanaerobacteraceae, Tepidanaerobacteraceae or Sporomusaceae,
genus Alkalibacillus, Bacillus, Geobacillus, Halobacillus, Lysinibacillus, Piscibacillus, Terribacillus, Brevibacillus, Paenibacillus, Thermobacillus, Pasteuria, Clostridium, Desulfotomaculum, Heliobacterium, Pelospora, Pelotomaculum, Caldanaerobacter, Moorella, Thermoanaerobacter, Tepidanaerobacter, Propionispora or Sporomusa, and
genus Bacillus, Paenibacillus or Clostridium.

15. A method of increasing or stabilising exopolysaccharide production or of reduction or prevention of exopolysaccharide degradation of a microorganism, comprising the step of providing, in the microorganism, the mutant DegS protein according to claim 3.

16. A method of increasing or stabilising exopolysaccharide production or of reduction or prevention of exopolysaccharide degradation of a microorganism, comprising the step of providing, in the microorganism, the mutant Spo0A protein according to claim 4.

17. An expression vector, comprising an expression cassette for expression of the mutant DegS protein according to claim 3.

18. An expression vector, comprising an expression cassette for expression of the mutant Spo0A protein according to claim 4.

19. The microorganism according to claim 1, comprising a mutant spo0A gene, wherein the mutation consists of or comprises A257S or I161L.

20. The microorganism according to claim 1, wherein the microorganism, when grown in a liquid fermentation medium, causes a viscosity increase of the fermentation medium such that the fermentation medium viscosity remains higher than 50% of the maximal fermentation medium viscosity obtained in a fermentation of the corresponding wild type strain over 48h after reaching the maximum carbon transfer rate (CTR) during a batch fermentation.
